(19) United States
(12) Patent Application Publication
(10) Pub. No.: US 2025/0058093 A1
(43) Pub. Date: Feb. 20, 2025

(54) SHUNT SYSTEM FOR DRAINING EXCESS CEREBROSPINAL FLUID

(71) Applicant: Cranial Devices Inc., Hummelstown, PA (US)

(72) Inventor: Barry M. FELL, Hummelstown, PA (US)

(21) Appl. No.: 18/809,241

(22) Filed: Aug. 19, 2024

Related U.S. Application Data
(60) Provisional application No. 63/520,232, filed on Aug. 17, 2023.
(60) Provisional application No. 63/588,762, filed on Oct. 9, 2023.

(51) Int. Cl. A61M 27/00

(52) U.S. Cl. CPC A61M 27/006; A61M 2205/3344

(57) ABSTRACT

A shunt system configured for draining excess cerebrospinal fluid (CSF) into a venous system of a patient includes an insertion assembly including a catheter, a catheter sheath at least partially surrounding a body of the catheter, and a stylet received in the catheter. An implant is coupled to the insertion assembly and disposed at the catheter distal end, the implant including an outer housing, an inner housing received within the outer housing, and a hollow needle extending from the inner housing and past a bottom surface of the outer housing. A guide plate is configured to receive the implant and be secured to the cranium over the burr hole, wherein the stylet tip protrudes past the needle distal end. The stylet and the catheter sheath are removable from the catheter such that the catheter is connectable to proximal shunt components for draining excess CSF into the venous system.

### CROSS-REFERENCE TO RELATED APPLICATIONS

This application claims the benefit of U.S. provisional application Ser. No. 63/520,232 filed Aug. 17, 2023 and U.S. provisional application Ser. No. 63/588,762 filed Oct. 9, 2023, the disclosures of which are hereby incorporated in their entirety by reference herein.

### TECHNICAL FIELD

Embodiments relate to a shunt system for draining excess cerebrospinal fluid into a venous system of a patient.

### BACKGROUND

When accessing the dura to implant a shunt system for draining excess cerebrospinal fluid, the surgeon must proceed very carefully so as to not risk ripping the sensitive dura. While one approach is to leave the inner table largely intact to protect the dura and drill directly over the sinus, the surgeon often does not know exactly where the sinus is as the information received from a navigation probe may not provide enough precision. The typical sagittal sinus of a human is generally “V”-shaped in cross-section. Finding the longitudinal center of the sinus is particularly important so that an implant is centered and does not accidentally pierce the interior sinus wall.

### SUMMARY

In one or more embodiments, a shunt system is configured for draining excess cerebrospinal fluid (CSF) into a venous system of a patient via a burr hole created in a cranium at a desired implant location. The shunt system includes an insertion assembly including a catheter having a proximal end and a distal end, a catheter sheath at least partially surrounding a body of the catheter between the catheter proximal end and the catheter distal end, and a stylet received in the catheter and having a tip. An implant is coupled to the insertion assembly and disposed at the catheter distal end, the implant including an outer housing, an inner housing received within the outer housing, and a hollow needle extending from the inner housing and past a bottom surface of the outer housing, the needle having a distal end. A guide plate is configured to receive the implant and be secured to the cranium over the burr hole, wherein the stylet tip protrudes past the needle distal end such that the needle distal end and the stylet tip are configured to penetrate a dura of a sagittal sinus exposed by the burr hole. The stylet and the catheter sheath are removable from the catheter such that the catheter is connectable to proximal shunt components for draining excess CSF into the venous system of the patient.

In one or more embodiments, the guide plate is configured to engage the outer housing to secure the implant to the guide plate. In one or more embodiments, the catheter distal end is connected to the inner housing. In one or more embodiments, the insertion assembly, the implant, and the guide plate are provided in a pre-assembled, sterile package. In one or more embodiments, the inner housing, the needle, and the guide plate each have a standard size, and wherein the outer housing is provided in different depths to accommodate for a specific thickness of the cranium of the patient at a desired implant location.

In one or more embodiments, the shunt system includes a depth gauge for aligning the guide plate with the desired implant location, wherein the depth gauge is configured to be removably received in the guide plate. In one or more embodiments, the depth gauge has a distal end with a guidance marker configured for aligning with a surgeon-placed aiming dot on the sagittal sinus, wherein a body of the depth gauge includes depth markings for determining a distance from the guide plate to the dura for selecting a suitable depth of the outer housing.

In one or more embodiments, the guide plate includes a generally planar top plate with a central cylindrical portion extending downwardly therefrom having a central lumen therethrough for receiving the outer housing. In one or more embodiments, the outer housing includes a flange extending outwardly therefrom, wherein the central cylindrical portion includes one or more inner recesses configured to engage the flange and secure the outer housing to the guide plate. In one or more embodiments, the guide plate includes a plurality of tabs with apertures for receiving fasteners to secure the guide plate to the cranium. In one or more embodiments, the guide plate includes a transverse channel for receiving and securing the catheter.

In one or more embodiments, the outer housing has a generally cylindrical body with a rounded bottom surface and a top surface having a cutout formed downwardly therefrom, wherein the cutout is configured to be aligned with the transverse channel for receiving the catheter. In one or more embodiments, the outer housing includes a plurality of arms terminating in apertures for receiving fasteners to secure the outer housing to the cranium. In one or more embodiments, the shunt system includes a guide plate holder configured to engage the guide plate to aid in placement of the guide plate during securing of the guide plate to the cranium.

In one or more embodiments, the inner housing includes a base portion and an upper portion, the upper portion having a smaller diameter than the base portion, wherein the catheter distal end is received on the upper portion. In one or more embodiments, the upper portion includes circumferential protrusions. In one or more embodiments, the stylet includes a body having a larger diameter than an end portion thereof, the body configured to abut a top end of the inner housing to limit an amount of the tip protruding past the needle distal end.

In one or more embodiments, the shunt system includes a catheter adapter disposed at the catheter proximal end. In one or more embodiments, the shunt system includes an adapter lock disposed on a distal portion of the catheter adapter and a proximal portion of the catheter sheath to secure the catheter adapter onto the catheter.

In one or more embodiments, the inner housing includes a surface-mounted sensor. In one or more embodiments, the sensor is provided on a top end of the inner housing and is configured for in vivo measurement of CSF pressure. In one or more embodiments, the sensor is provided on a base portion of the inner housing and is configured to contact the dura for in vivo measurement of sinus pressure.

In one or more embodiments, the needle is retractable and extendable with respect to the inner housing. In one or more embodiments, engagement of the stylet with the inner housing causes the needle to be extended from the inner housing. In one or more embodiments, the shunt system includes a removable cover for the needle distal end.

In one or more embodiments, a shunt system is configured for draining excess cerebrospinal fluid (CSF) into a venous system of a patient via a burr hole created in a cranium at a desired implant location. The shunt system includes an insertion assembly including a catheter having a proximal end and a distal end, a catheter sheath at least partially surrounding a body of the catheter between the catheter proximal end and the catheter distal end, and a stylet received in the catheter and having a tip. An implant is coupled to the insertion assembly and disposed at the catheter distal end, the implant including an outer housing including a flange extending outwardly therefrom, an inner housing received within the outer housing, and a hollow needle extending from the inner housing and past a bottom surface of the outer housing, the needle having a distal end. A guide plate is configured to receive the implant and be secured to the cranium over the burr hole, the guide plate including one or more inner recesses configured to engage the flange to secure the outer housing to the guide plate, wherein the stylet tip protrudes past the needle distal end such that the needle distal end and the stylet tip are configured to penetrate a dura of a sagittal sinus exposed by the burr hole. The stylet and the catheter sheath are removable from the catheter such that the catheter is connectable to proximal shunt components for draining excess CSF into the venous system of the patient.

In one or more embodiments, a shunt system is configured for draining excess cerebrospinal fluid (CSF) into a venous system of a patient via a burr hole created in a cranium at a desired implant location. The shunt system includes an insertion assembly including a catheter having a proximal end and a distal end, a catheter sheath at least partially surrounding a body of the catheter between the catheter proximal end and the catheter distal end, and a stylet received in the catheter and having a tip. An implant is coupled to the insertion assembly and disposed at the catheter distal end, the implant including an outer housing configured to be secured directly to the cranium, an inner housing received within the outer housing, and a hollow needle extending from the inner housing and past a bottom surface of the outer housing, the needle having a distal end. A guide plate is configured to receive the implant and be secured to the cranium over the burr hole, wherein the stylet tip protrudes past the needle distal end such that the needle distal end and the stylet tip are configured to penetrate a dura of a sagittal sinus exposed by the burr hole. The stylet and the catheter sheath are removable from the catheter such that the catheter is connectable to proximal shunt components for draining excess CSF into the venous system of the patient.

In one or more embodiments, a method for draining excess cerebrospinal fluid (CSF) into a venous system of a patient via a burr hole created in a cranium at a desired implant location includes providing an insertion assembly including a catheter having a proximal end and a distal end, a catheter sheath at least partially surrounding a body of the catheter between the catheter proximal end and the catheter distal end, and a stylet received in the catheter and having a tip. The method further includes providing an implant coupled to the insertion assembly and disposed at the catheter distal end, the implant including an outer housing, an inner housing received within the outer housing, and a hollow needle extending from the inner housing and past a bottom surface of the outer housing, the needle having a distal end, wherein the stylet tip protrudes past the needle distal end. The method further includes receiving the implant in a guide plate, securing the guide plate to the cranium over the burr hole, penetrating a dura of a sagittal sinus exposed by the burr hole with the needle distal end and the stylet tip, removing the stylet and the catheter sheath from the catheter, and connecting the catheter to proximal shunt components for draining excess CSF into the venous system of the patient.

In one or more embodiments, the method includes marking the desired implant location on the sagittal sinus with an aiming dot. In one or more embodiments, the method includes inserting a depth gauge into the guide plate and aligning a guidance marker of the depth gauge with the aiming dot. In one or more embodiments, the method includes using depth markings on the depth gauge to select the outer housing with a suitable depth based on a thickness of the cranium at the desired implant location.

In one or more embodiments, the method includes placing the guide plate over the burr hole using a guide plate holder. In one or more embodiments, the method includes rotating the insertion assembly to secure the implant to the guide plate. In one or more embodiments, rotating the insertion assembly to secure the implant to the guide plate includes engaging a flange of the outer housing with an inner recess of the guide plate.

In one or more embodiments, the method includes securing the outer housing directly to the cranium. In one or more embodiments, the method includes securing the catheter in the guide plate. In one or more embodiments, securing the catheter in the guide plate includes receiving the catheter in a transverse channel of the guide plate. In one or more embodiments, receiving the implant in the guide plate includes aligning a cutout of the outer housing with the transverse channel of the guide plate.

### BRIEF DESCRIPTION OF THE DRAWINGS

FIG. 1 is a perspective view of an insertion assembly and an implant assembly of a shunt system according to one or more embodiments;

FIG. 2 is an exploded view of the insertion assembly and implant assembly of FIG. 1;

FIG. 3 illustrates a top view of the cranium with a burr hole therethrough to expose the sagittal sinus for midline placement of the guide plate according to one or more embodiments;

FIG. 4 is a medial-lateral cross-section of the cranium illustrating the burr hole, the sagittal sinus, and a location of an aiming dot drawn on the sagittal sinus for use with a depth gauge for positioning of the guide plate according to one or more embodiments;

FIG. 5 illustrates a top view of the cranium as shown in FIG. 3 with the guide plate in position over the burr hole;

FIG. 6 is a perspective view of the depth gauge prior to engagement with the guide plate according to one or more embodiments;

FIG. 7 is a perspective view of the depth gauge engaged with and extending through the guide plate;

FIG. 8 is a top view of the cranium illustrating location of the midline of the sagittal sinus and placement of the guide plate using the depth gauge;

FIG. 9 is a cross-sectional view of the cranium illustrating location of the midline of the sagittal sinus and placement of the guide plate using the depth gauge;

FIG. 10 is a perspective view of a guide plate holder prior to engagement with the guide plate according to one or more embodiments;

FIG. 11 is a perspective view of the guide plate holder engaged with the guide plate;

FIG. 12 is a top view of the guide plate holder engaged with the guide plate;

FIG. 13 is a perspective view of an outer housing of an implant according to one or more embodiments;

FIG. 14 is a front view of the outer housing of FIG. 13;

FIG. 15 is a bottom view of the outer housing of FIG. 13;

FIG. 16 is a perspective view of an inner housing of the implant according to one or more embodiments;

FIG. 17 is a front view of the inner housing of FIG. 16;

FIG. 18 is a front view of the inner housing and the needle thereof;

FIG. 19 is a perspective view of the assembled implant including the inner housing received in the outer housing, with a distal end of the needle extending therethrough according to one or more embodiments;

FIG. 20 is a front view of the implant of FIG. 19;

FIG. 21 is a bottom view of the implant of FIG. 19;

FIG. 22 is a front view of the shunt system including the insertion assembly with the catheter sheath and the implant assembly according to one or more embodiments;

FIG. 23 is a front view of the shunt system of FIG. 22 without the catheter sheath;

FIG. 24 is an enlarged view of a proximal portion of the insertion assembly and stylet;

FIG. 25 is a cross-sectional view of the proximal portion of FIG. 24;

FIG. 26 is a cross-sectional view of the implant assembly and a distal portion of the insertion assembly according to one or more embodiments;

FIG. 27 is a cross-sectional view of the implant assembly and a distal portion of the insertion assembly with the catheter removed;

FIG. 28 is an enlarged view illustrating the stylet tip extending from the distal end of the inner housing;

FIG. 29 illustrates the distal portion of the insertion assembly and implant prior to insertion into the guide plate;

FIG. 30 illustrates the distal portion of the insertion assembly and the implant assembly with the outer housing of the implant received in the guide plate and the needle distal end and the stylet tip extending below the guide plate;

FIG. 31 is a top perspective view of the distal portion of the insertion assembly and the implant inserted through the guide plate with a tab of the outer housing aligned with a transverse channel of the guide plate such that the needle distal end is positioned for penetrating the dura and extending into the sinus according to one or more embodiments;

FIG. 32 is a top perspective view of the distal portion of the insertion assembly and the implant illustrating locking to the guide plate by twisting the insertion assembly so that the outer housing tab engages an inner recess of a central cylindrical portion of the guide plate;

FIG. 33 is a top perspective view of the distal portion of the insertion assembly and the implant with the tab rotated approximately 90 degrees from the transverse channel at which point the implant is fully locked to the guide plate and the catheter sheath can be removed;

FIG. 34 is a top perspective view of the catheter and the implant locked to the guide plate once the catheter sheath has been removed according to one or more embodiments;

FIG. 35 is a top perspective view of the cranium with the guide plate and implant secured thereto after the stylet is removed, wherein the catheter is held within the arms of the transverse channel of the guide plate according to one or more embodiments;

FIG. 36 is a top perspective view of the guide plate, the implant, and the secured catheter;

FIG. 37 is a rear perspective view of the guide plate, the implant, and the secured catheter;

FIG. 38 is a cross-sectional view of the guide plate, the implant with the catheter secured to the top portion of the inner housing and the outer housing secured to the guide plate, and the catheter held in the transverse channel of the guide plate after the stylet has been removed;

FIG. 39 is a front view of the guide plate, received implant, and secured catheter;

FIG. 40 is a perspective view of the inner housing illustrating an embodiment wherein an integrated, surface-mounted sensor is provided on a top portion of the inner housing for in vivo measurement of cerebrospinal fluid (CSF) pressure;

FIG. 41 is a top view of the inner housing within the outer housing illustrating an embodiment wherein an integrated, surface-mounted sensor is provided on a base of the inner housing and configured to contact the dura for in vivo measurement of sinus pressure;

FIG. 42 is a side view of a catheter sheath that eliminates the adapter lock and replaces it with a breakaway element joined between the sheath and the catheter adapter according to one or more embodiments;

FIG. 43 is a perspective view of the catheter sheath of FIG. 42 attached to the catheter adapter;

FIG. 44 is a cutaway, front view of the catheter sheath of FIG. 42 illustrating a gap between the catheter adapter and the sheath;

FIG. 45 shows a cross-sectional view of the cranium and the superior sagittal sinus, and illustrates a first step in the surgical method according to one or more embodiments, including removing cranial bone starting lateral of the sagittal sinus down to the inner table, and removing the inner table and progressing medially until the midpoint of the sagittal sinus is exposed, forming a burr hole or slot in the cranium;

FIG. 46A shows a top view of the slot in the cranium and the sinus, and illustrates a continuation of the first surgical step according to one or more embodiments, including locating the center of the sinus and marking the intended implant location;

FIG. 46B is an enlarged view of the indicated area of FIG. 46A;

FIG. 47 shows a perspective view of the guide plate holder and guide plate, and illustrates a second step in the surgical method according to one or more embodiments, including placing the guide plate into the slot in the cranium using the guide plate holder;

FIG. 48 shows a perspective view of the depth gauge prior to insertion into the guide plate holder and guide plate, and illustrates a continuation of the second surgical step according to one or more embodiments, including inserting the depth gauge through the guide plate holder and into the guide plate;

FIG. 49 shows a top view of the guide plate holder and guide plate with the depth gauge inserted, and illustrates a third step in the surgical method according to one or more embodiments, including aligning a center mark of the depth gauge with the surgeon-placed mark indicating the longitudinal axial centerline of the sinus;

FIG. 50 shows a perspective view of the depth gauge received in the guide plate, and illustrates a fourth step in the surgical method according to one or more embodiments, including screwing down the guide plate using flush, low-profile CMF bone screws through tabs in the guide plate, ensuring that the guide plate stays centered on the sinus;

FIG. 51 shows the guide holder removed from the guide plate with the depth gauge remaining in the guide plate, and illustrates a continuation of the fourth surgical step according to one or more embodiments, including removing the guide plate holder once the guide plate is secured to the cranium;

FIG. 52 is a front view of a depth gauge having a plurality of depth markings according to one or more embodiments;

FIG. 53 is a front view of the depth gauge inserted in the guide plate, and illustrates a fifth step in the surgical method according to one or more embodiments wherein the depth gauge is used to determine the sizing of the outer housing of the implant;

FIG. 54 is a side view of the insertion assembly including a Tuohy Borst adapter with a port for saline engaged with the catheter adapter, and illustrates a sixth step in the surgical method according to one or more embodiments wherein the outer housing of the implant is selected as indicated by the depth markings of the depth gauge, and the insertion assembly is flushed with sterile saline;

FIG. 55 is a perspective view of the insertion assembly and implant prior to insertion into the guide plate, and illustrates a seventh step in the surgical method according to one or more embodiments wherein the properly sized, sterile saline-filled insertion assembly and implant are inserted into the guide plate;

FIG. 56 is an enlarged view of the implant received in the guide plate with the outer housing tab aligned with the transverse channel of the guide plate, and illustrates a continuation of the seventh surgical step according to one or more embodiments wherein the insertion assembly and implant are inserted into the guide plate with the outer housing tab aimed toward the transverse channel of the guide plate;

FIG. 57 is a cross-sectional view of the insertion assembly and implant received in the guide plate with the needle distal end in the sinus, and illustrates an eighth step of the surgical method according to one or more embodiments wherein the insertion assembly is inserted into the guide plate until the outer housing of the implant contacts the bottom of the burr slot and the stylet penetrates the dura into the sinus;

FIG. 58 is a perspective view of the insertion assembly received in the guide plate, and illustrates a continuation of the eighth surgical step according to one or more embodiments, wherein the insertion assembly is rotated approximately 90 degrees to secure the implant to the guide plate;

FIG. 59 is a perspective view of the insertion assembly with the stylet removed, and illustrates a ninth step of the surgical method according to one or more embodiments including attaching a manometer to the Tuohy Borst adapter and filling the insertion assembly with saline, then removing the stylet and observing a drop in the manometer showing patency and fluid movement through the implant into the sagittal sinus;

FIG. 60 is a perspective view of the insertion assembly, and illustrates a tenth step of the surgical method according to one or more embodiments, including rotating an adapter lock to an open position once patency has been demonstrated to allow for removal of the catheter sheath;

FIG. 61 is a perspective view of the insertion assembly with the catheter sheath removed, and illustrates a continuation of the tenth surgical step, including removing the catheter sheath from the insertion assembly, wherein the adapter lock, catheter adapter, and Tuohy Borst adapter may also now be removed;

FIG. 62 is an enlarged view of the cranium showing the guide plate, secured catheter, and proximal shunt components, and illustrates an eleventh step in the surgical method according to one or more embodiments, including bending the catheter over and into the transverse channel in the guide plate and attaching the catheter to proximal shunt components;

FIG. 63 is a perspective view of the cranium illustrating the guide plate, secured catheter, and connected proximal shunt components including a one way valve, proximal catheter, and burr hole reservoir;

FIG. 64 is a perspective view of the cranium, dura, and sagittal sinus receiving an implant, connected catheter, and guide plate according to another embodiment;

FIG. 65 is a top view of the guide plate of the embodiment of FIG. 64;

FIG. 66 is a front view of the guide plate of the embodiment of FIG. 64;

FIG. 67 is a cross-sectional view of the guide plate of the embodiment of FIG. 64;

FIG. 68 is a top view of an implant of the embodiment of FIG. 64, illustrating the outer housing and the inner housing;

FIG. 69 is a cutaway perspective view of the assembled implant of the embodiment of FIG. 64 with a distal end of a catheter attached to the inner housing (not shown);

FIG. 70 is a cross-sectional view of the assembled implant of the embodiment of FIG. 64 with the catheter attached to the inner housing;

FIG. 71 is a top view of the assembled guide plate and implant of the embodiment of FIG. 64;

FIG. 72 is a side view of the assembled guide plate and implant of FIG. 71 with a catheter attached to the inner housing;

FIG. 73 is a cross-sectional view of the assembled guide plate and implant of FIG. 71 with a catheter attached to the inner housing, illustrating an embodiment with an optional extendable needle distal end shown in a retracted position;

FIG. 74 is a cross-sectional view of the assembled guide plate and implant of FIG. 73 with the needle distal end shown in an extended position below the inner housing;

FIG. 75 is a side view of the assembled guide plate, implant, and catheter of FIG. 72 with the catheter received in the guide plate;

FIG. 76 is a cross-sectional view of the assembled guide plate and implant of FIG. 73 with the inner housing positioned on the dura prior to extension of the extendable needle distal end;

FIG. 77 is a cross-sectional view of the assembled guide plate and implant of FIG. 74 with the inner housing positioned on the dura and the needle distal end extended below the inner housing and into the sagittal sinus;

FIG. 78 is a cross-sectional view of the assembled guide plate and implant of FIG. 75 with the inner housing positioned on the dura, the needle distal end extended below the inner housing and into the sagittal sinus, and the catheter received in the guide plate;

FIG. 79 is a perspective view of an insertion assembly and implant according to the embodiment of FIG. 64 prior to engagement with the guide plate positioned on the cranium;

FIG. 80 is a perspective view of the implant assembly of FIG. 79 engaged with the guide plate positioned on the cranium;

FIG. 81 is a perspective view of the insertion assembly of FIG. 80 engaged with the guide plate and illustrating removal of the stylet from the catheter;

FIG. 82 is a cross-sectional view of a proximal portion of the insertion assembly according to an embodiment including an adapter body having a distal end connected to a proximal end of the catheter, a side port configured to receive saline-filled tubing, and a proximal end for receiving an adapter lock, the adapter body including a stylet seal, and a stylet received through the adapter lock, the stylet seal, and the adapter body to extend into the catheter, with a catheter sheath at least partially encircling the catheter;

FIG. 83 is a side view of the insertion assembly of FIG. 82;

FIG. 84 is a cross-sectional view of a distal portion of the insertion assembly and the assembled guide plate and implant of FIG. 77 with a stylet inserted in the catheter but retracted proximal of the needle distal end;

FIG. 85 is a cross-sectional view of the assembled guide plate and implant of FIG. 77 with the stylet inserted in the catheter and advanced to extend through the needle distal end;

FIG. 86 is a perspective view of a distal portion of the insertion assembly and implant according to the embodiment of FIG. 64 prior to insertion into the guide plate;

FIG. 87 is a perspective view of the insertion assembly and implant of FIG. 86 after insertion into the guide plate, with the adapter body, stylet, and saline-filled tube removed for clarity;

FIG. 88 is a perspective view of the implant of FIG. 86 with the guide plate and outer housing secured to the cranium and the catheter received in the guide plate;

FIG. 89 is a side view of an insertion assembly and implant according to the embodiment of FIG. 79;

FIG. 90 is an exploded view of the insertion assembly and implant of FIG. 89 along with a guide plate;

FIG. 91 is a side view of the insertion assembly and implant of FIG. 89 with a protective cover for the needle distal end; and

FIG. 92 is a perspective view of the insertion assembly and implant of FIG. 89 illustrating removal of the catheter sheath from the catheter and adapter body.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Embodiments disclosed herein include a shunt system for restoring the natural physiologic pathway for excess cerebrospinal fluid (CSF) to drain from the ventricles to the venous system (superior sagittal sinus) of the body. The shunt system disclosed herein eliminates the need for a long-length distal catheter tube, eliminates gravity-induced siphoning of CSF, reduces the chance of clogging of the ventricular catheter (no suction effect), and is optimized for single-surgeon operation.

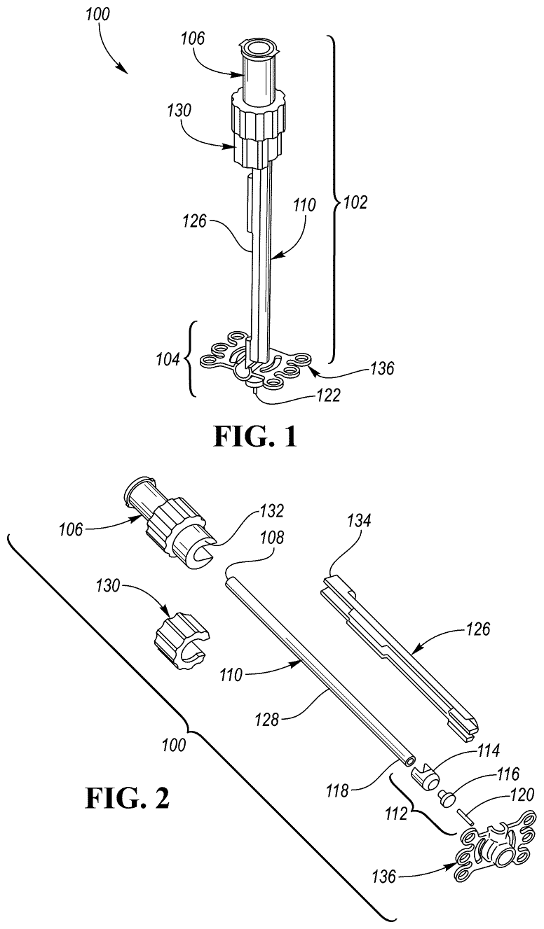

FIGS. 1 and 2 illustrate components of the shunt system 100 according to one or more embodiments including an insertion assembly 102 and an implant assembly 104. The insertion assembly 102 may include a catheter adapter 106 (e.g. plastic) positioned on a proximal end 108 of a catheter 110. The implant assembly 104 may include an implant 112 including an outer housing 114 (e.g. PEEK) and an inner housing 116 (e.g. PEEK) disposed at a distal end 118 of the catheter 110. A needle 120 may be received in the inner housing 116 at the distal end 118 of the catheter 110, and is the projecting, hollow, rigid lumen portion of the assembled implant 112. The needle 120 has a distal end 122, wherein a penetrating stylet 124 (shown in subsequent figures) is configured to protrude past the needle distal end 122 to facilitate insertion through the superior dura D surface of the sagittal sinus S and into the venous bloodstream. A catheter sheath 126 (e.g. plastic) may at least partially surround a body 128 of the catheter 110 between the proximal end 108 and the distal end 118, and an adapter lock 130 (e.g. plastic) may be coupled to the catheter adapter 106 (e.g. disposed on a distal portion 132 of the catheter adapter 106 and a proximal portion 134 of the catheter sheath 126) to secure the catheter adapter 106 onto the catheter 110. The implant assembly 104 includes the implant 112 and a guide plate 136 (e.g. titanium), and in one or more embodiments the insertion assembly 102 and the implant 112 are configured to be received in the guide plate 136.

In one or more embodiments, the shunt system 100 may be provided to the surgeon in a pre-assembled, sterile package. The inner housing 116, the hollow needle 120, and the guide plate 136 may be common for or of standard size in all implant assemblies 104, with the variable component being the outer housing 114, specifically the depth dimension thereof. The guide plate 136 is configured to sit on the outer surface of the cranium C, called the outer table, where a patient may have only a 5 mm thick cranium or perhaps a 10 mm thick cranium, for example. In the shunt system 100 shown and described herein, advantageously the only variable that the surgeon needs to allow for is the depth of the outer housing 114 (the only modular piece that is necessary for the shunt system 100) to accommodate specifically for the patient's cranium thickness at the desired implant location. Thus, a range of cranium thicknesses can be accommodated by selecting an appropriate depth outer housing 114, from a provided library of outer housings 114 of different depths, in order to fit a range of cranium thicknesses in different patients.

The shunt system 100 as shown and described herein may be provided to the surgeon fully assembled (including the stylet 124). The coupling of the needle 120 to the catheter 110 (e.g. via the inner housing 116 and outer housing 114) surrounded by the catheter sheath 126 which is, in turn, rigidly attached to the catheter adapter 106 at a predetermined length, and into which the rigid, solid stylet 124 is disposed, all combines to form a long, rigid insertion assembly 102 and implant 112 with a very sharp tip 137 that will comfortably fit into a surgeon's hand and fingers, much like a pencil or pen is typically held. The combination of the stiffness and length of the insertion assembly 102, as well as the comfortable, precise fit, allows the surgeon to experience maximum tactile feedback of the operative method when placing the implant 112 from the time of first engagement of the dura D over the sagittal sinus S to the subsequent penetration of the dura D. This tactile engagement, often described as a “pop,” is familiar to any physician who is experienced with dural access, whether it be during a spinal tap or cranial access. It is important to note that the “pop” only occurs when a needle is placed in a linear traveling manner through a dura layer of tissue (i.e. with no rotation), and this “pop” is not present or is only minimally perceptible if a threaded, advancing rotating needle is used instead as is the case in some prior art devices.

When accessing the dura D to implement a shunt system 100 for draining excess CSF, a surgeon may wish to begin burring into the cranium C with a lateral or sideways trajectory until they reach the dura D, then work their way toward the sagittal sinus S where they will observe a color change. The surgeon can then confirm the dimensions of the sagittal sinus S via direct visualization aided with navigated imaging via CT or MRI, and ensure that they are centered over the longitudinal axis of the sagittal sinus S before proceeding further with implantation.

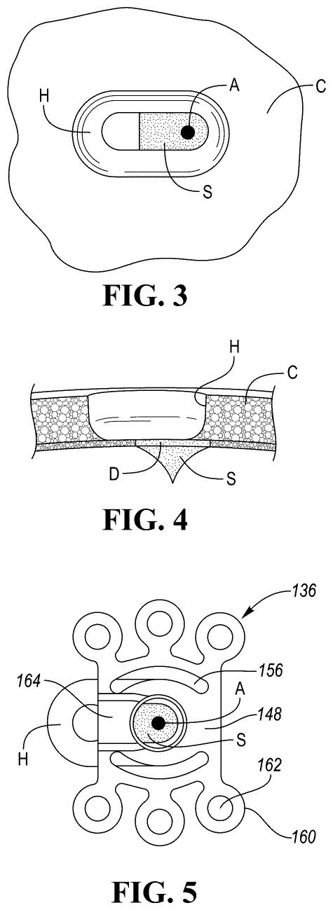

FIGS. 3-5 illustrate a burr hole H (or slot) through the cranium C and exposure of the sagittal sinus S for midline placement of the guide plate 136 according to one or more embodiments. FIGS. 3 and 4 illustrate the burr hole H, the exposed sagittal sinus S (axial view), and an aiming dot A on the sagittal sinus S for aligning with a depth gauge 138 such as an optical aiming scope or another depth gauge or guide (discussed further below) for positioning the guide plate 136. FIG. 5 illustrates a top view of the cranium C as shown in FIG. 3 with the guide plate 136 in position over the burr hole H. As described above, based on imaging, the surgeon may select a point to begin burring (e.g. with a 4 mm burr) which is not directly over the sagittal sinus S so as not to inadvertently hit the sagittal sinus S. As the surgeon burrs down into the cranium C, they will be able to see the edge of the sagittal sinus S and confirm this location with imaging. The surgeon can then determine the midline of the sagittal sinus S, visually or combined with typical imaging navigational tools, continue creating the burr hole H so that it is over the midline, and then indicate the longitudinal midpoint with an aiming dot A, such as with a methylene blue pen. At this point, the guide plate 136 may be centered over the midpoint (aiming dot A).

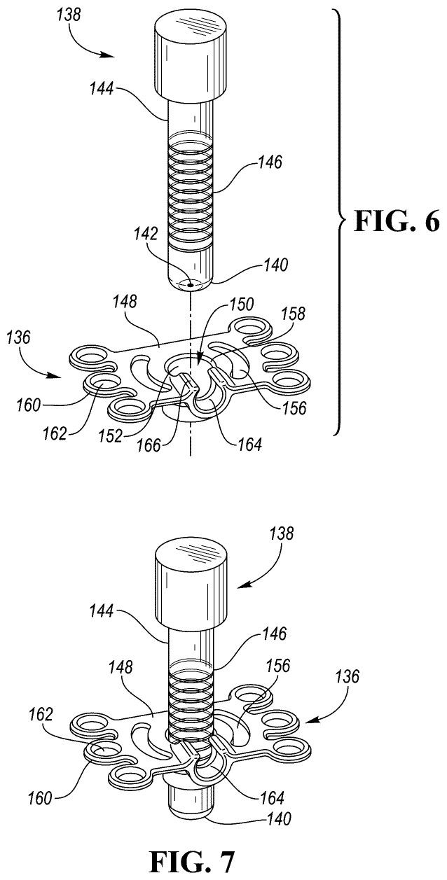

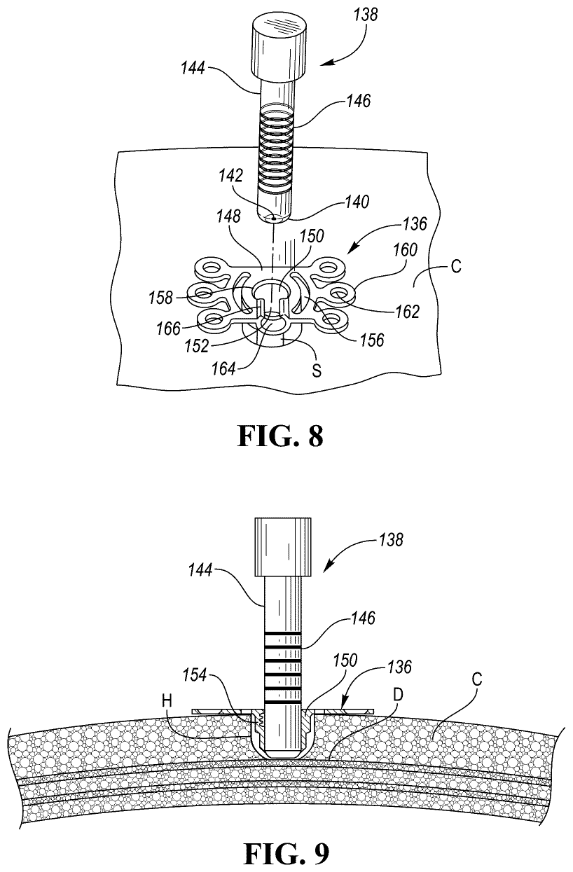

FIGS. 6-9 illustrate a depth gauge 138 used for properly positioning the guide plate 136 over the desired portion of the sagittal sinus S according to one or more embodiments. FIG. 6 is a perspective view of the depth gauge 138 prior to engagement with the guide plate 136, and FIG. 7 is a perspective view of the depth gauge 138 engaged with and extending through the guide plate 136. FIG. 8 is a top view and FIG. 9 is a cross-sectional view of the cranium C illustrating location of the midline of the sagittal sinus S and placement of the guide plate 136 using the depth gauge 138. In one or more embodiments, the depth gauge 138 may be constructed from transparent plastic, with a distal end 140 having a guidance marker 142 for aligning with the aiming dot D marked on the previously exposed longitudinal axis of the sagittal sinus S. A shaft or body 144 of the depth gauge 138 may include depth markings 146 for determining the distance from the guide plate 136 to the dura D. This distance may be used in selecting the appropriate outer housing 114 of the implant 112, as described further below. While a particular configuration of the depth gauge 138 is shown and described herein, it is understood that alternative depth gauges may also be possible.

In one or more embodiments, the guide plate 136 includes a generally planar top plate 148 with a central cylindrical portion 150 extending downwardly therefrom with a central lumen 152 therethrough. As best shown in subsequent figures, the central cylindrical portion 150 includes one or more inner recesses 154 which serve as engagement areas with the implant 112, such as the outer housing 114. The top plate 148 may include slots 156 on either side of a top end of the central cylindrical portion 150, and a plurality of tabs 160 with apertures 162 for receiving fasteners 216 (e.g. screws) to secure the guide plate 136 to the cranium C. A transverse channel 164 may be formed in communication with the central lumen 152 and may include opposed arms 166 extending upwardly above the top plate 148. Exemplary dimensions of the guide plate 136 may be approximately 20 mm in length and 14 mm in width. Of course, it is understood that the guide plate 136 is not limited to the particular configuration or dimensions described above. The structure and functions of the guide plate 136 will be described further below.

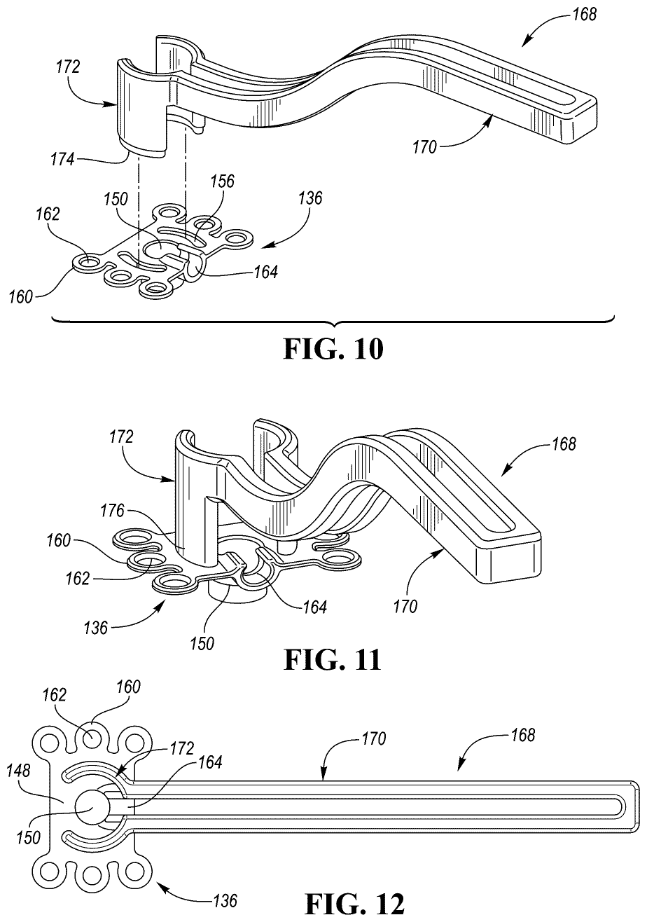

FIGS. 10-12 illustrate a guide plate holder 168 configured to engage the guide plate 136 to aid in placement of the guide plate 136 during its fixation to the cranium C. FIG. 10 shows the guide plate holder 168 prior to engagement with the guide plate 136, and FIGS. 11 and 12 are perspective and top views, respectively, of the guide plate holder 168 engaged with the guide plate 136. In one or more embodiments, the guide plate holder 168 may be constructed from a plastic material, and may include a handle portion 170 and a distal head portion 172. The handle portion 170 may be curved such as, but not limited to, with the shape depicted herein to accommodate the surgeon's fingers underneath. In one or more embodiments, the distal head portion 172 may include two spaced flanges 174 extending downwardly below the handle portion 170 at a distal end 176 of the guide plate holder 168, wherein the flanges 174 are configured to engage the slots 156 in the guide plate 136 in order to hold the guide plage 136 in place on the cranium C. While the flanges 174 and the corresponding slots 156 are shown herein to be arcuate in shape, it is understood that other configurations are also possible. The flanges 174 may be configured to be received in the guide plate 136 with an interference fit. Although not shown, the depth gauge 138 may be used while the guide plate holder 168 is stabilizing the guide plate 136 to ensure that the guide plate 136 remains in the desired position on the cranium C while being secured with fasteners 216, wherein this process may include aligning the guidance marker 142 on the depth gauge 138 with the aiming dot A on the sagittal sinus S.

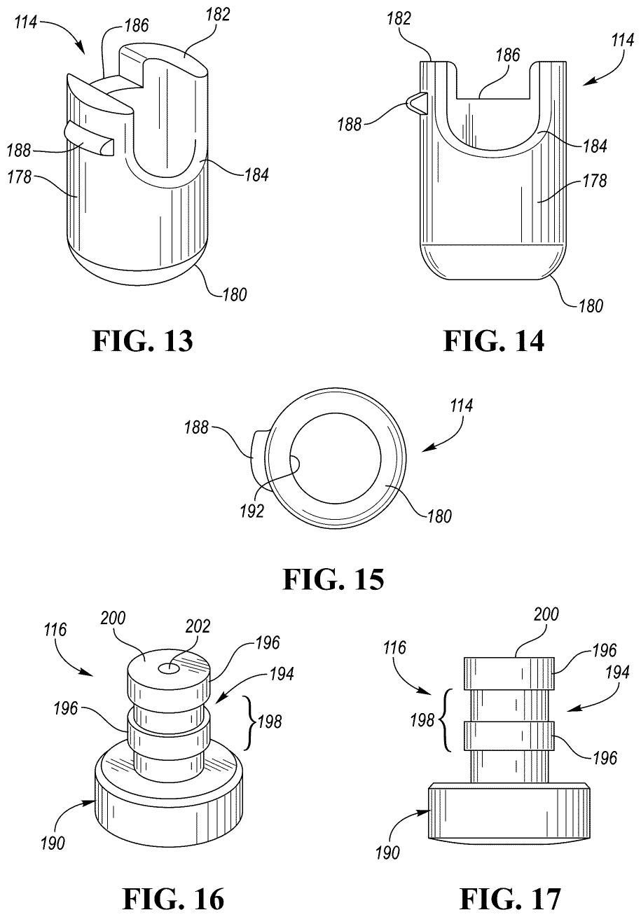

FIGS. 13-15 illustrate views of the outer housing 114 of the implant 112 according to one or more embodiments. The outer housing 114 may have a generally cylindrical body 178 with a rounded bottom surface 180. In one or more embodiments, the shape of the bottom surface 180 may be complementary to that of a 4 mm burr, for example. The top surface 182 may be asymmetrical, with a rounded cutout 184 formed downwardly from the top surface 182 and an opposed smaller ledge 186 formed downwardly from the top surface 182. A flange 188 may extend outwardly from the outer housing 114 on one side of the body 178. The outer housing 114 may be provided in a plurality of different depths (for example, but not limited to, 5.5 mm, 7.0 mm, 8.5 mm, 10.0 mm, 11.5 mm, and 13.0 mm), the most suitable of which may be selected depending on the thickness of the patient's cranium C as described further below.

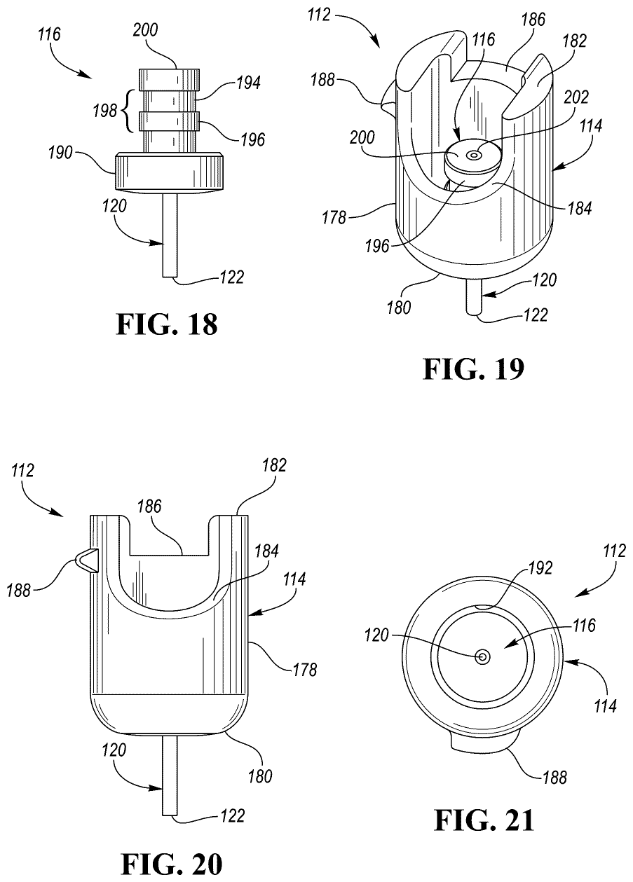

FIGS. 16-18 illustrate views of the inner housing 116 of the implant 112 according to one or more embodiments, and FIGS. 19-21 illustrate views of the assembled implant 112 with the inner housing 116 received within the body 178 of the outer housing 114, wherein the inner housing 116 may be press fit or adhered/bonded to the outer housing 114. The inner housing 116 may include a cylindrical base portion 190 arranged to abut or engage an interior bottom surface 192 of the outer housing 114. An upper portion 194 of the inner housing 116 has a smaller diameter than the base portion 190, and includes circumferential protrusions 196 in a middle region 198 and top end 200 of the upper portion 194. In one or more embodiments, the catheter distal end 118 is configured to be received on the upper portion 194 of the inner housing 116, such as by stretching the distal end 118 over the protrusions 196, adhesively bonding the catheter 110 to the inner housing 116, and/or attaching the catheter 110 to the inner housing 116 with suture. The inner housing 116 has a lumen 202 therethrough, and the needle 120 extends downwardly from the base portion 190 and past the bottom surface 180 of the outer housing 114 when the inner housing 116 is received in the outer housing 114. It is understood that although particular configurations of the outer housing 114 and the inner housing 116 are shown and described herein, other configurations of these components are also contemplated which may be assembled and cooperate to form the implant 112.

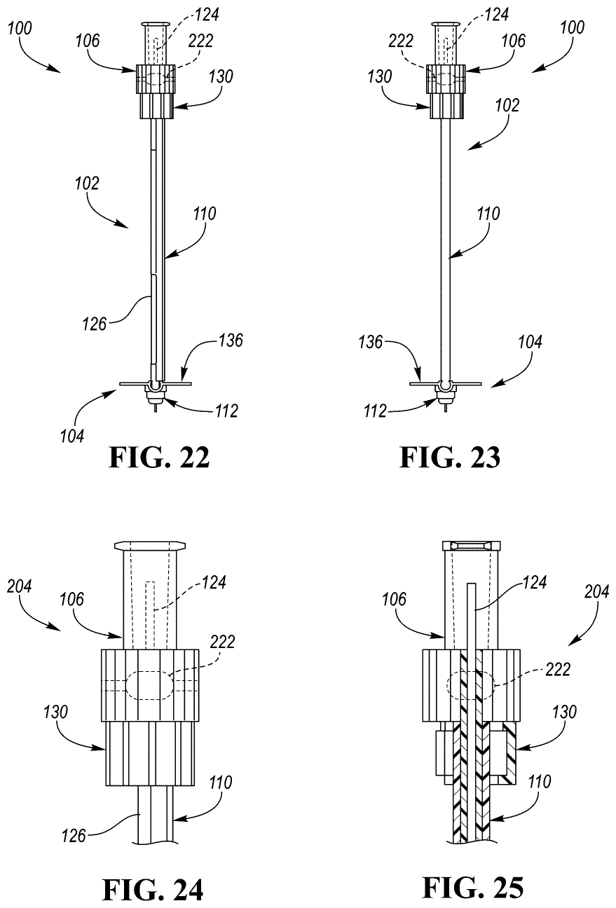

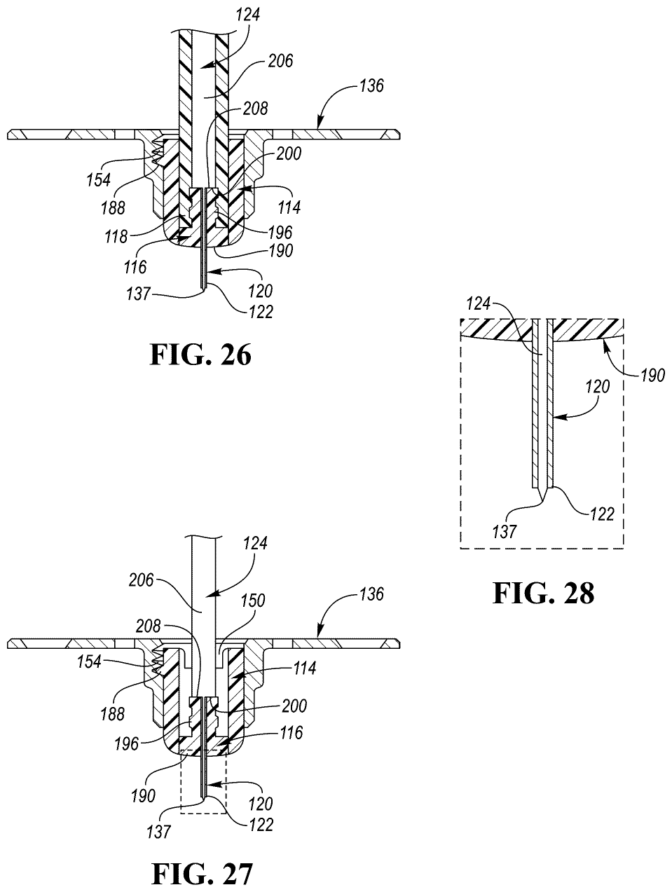

FIG. 22 is a front view of the insertion assembly 102 and the implant assembly 104 according to one or more embodiments, FIG. 23 is a front view of the insertion assembly 102 of FIG. 22 without the catheter sheath 126, FIG. 24 is an enlarged view of a proximal portion 204 of the insertion assembly 102 and the stylet 124, and FIG. 25 is a cross-sectional view of the proximal portion 204 of the insertion assembly 102. In the assembled shunt system 100 the internal, solid-tipped, sharp stylet 124 fits inside the catheter 110 and through the distal end 122 of the hollow needle 120. As best shown in FIGS. 26 and 27, there may be an internal integrated depth stop for the stylet 124 created by the inner housing 116. More particularly, the stylet 124 may include a body 206 having a larger diameter than an end portion 208 thereof. When the stylet 124 is advanced through the catheter 110, the stylet body 206 will abut the top end 200 of the inner housing 116 such that the distal placement of the stylet 124 is halted with the amount of exposed sharpened stylet tip 137 coming through the distal end 122 of the hollow needle 120 limited to a desired amount of protrusion, typically less than 1 mm, for example.

FIG. 26 is a cross-sectional view of a distal portion of the shunt system 100 according to one or more embodiments. In this illustration, the outer housing 114 is engaged with and locked to the guide plate 136 and the needle distal end 122 and the stylet tip 137 are in position to penetrate the dura D. As described further below, the implant 112 has been rotated approximately 90 degrees from an insertion position so that the flange 188 of the outer housing 114 engages one of the plurality of inner recesses 154 in the cylindrical portion 150 of the guide plate 136 to lock the outer housing 114 to the guide plate 136. In one or more embodiments, the recesses 154 are spaced approximately 0.5 mm apart, such that the option of the flange 188 engaging any one of the recesses 154 serves as a final adjustment mechanism for the depth of the implant 112. FIG. 27 is a cross-sectional view of the distal portion of the shunt system 100 with the catheter 110 removed for clarity to better demonstrate the design of the stylet 124 and its integral depth stop shape in contact with the top end 200 of the inner housing 116, as described above. FIG. 28 is an enlarged view illustrating the stylet tip 137 extending from the needle distal end 122.

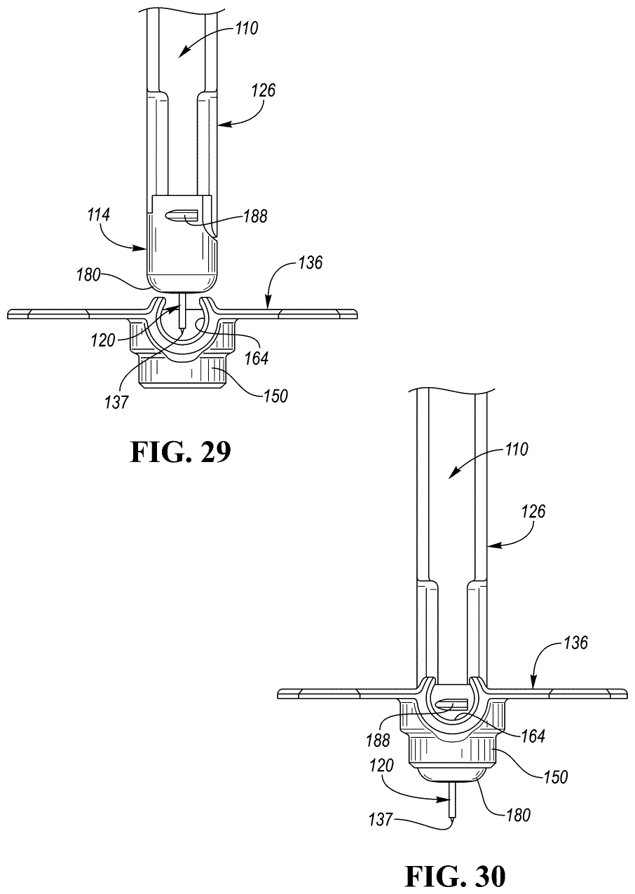

The implant 112 is coupled to the insertion assembly 102 via the catheter 110, with the outer housing 114 adjacent to and/or abutting the catheter sheath 126. FIG. 29 illustrates the distal portion of the insertion assembly 102 and the implant 112 prior to insertion into the guide plate 136, and FIG. 30 illustrates the distal portion of the insertion assembly 102 with the implant 112 received in the guide plate 136 and the needle distal end 122 and the stylet tip 137 extending below the guide plate 136. In one or more embodiments, during insertion of the implant 112 into the guide plate 136, the flange 188 of the outer housing 114 should be aligned with the transverse channel 164 of the guide plate 136 in order for the implant 112 to be substantially received in the guide plate cylindrical portion 150. In this position, the outer housing 114 (e.g. bottom surface 180) will sit on the dura D with the needle distal end 122 and the stylet tip 137 having penetrated the dura D (or in position to do so).

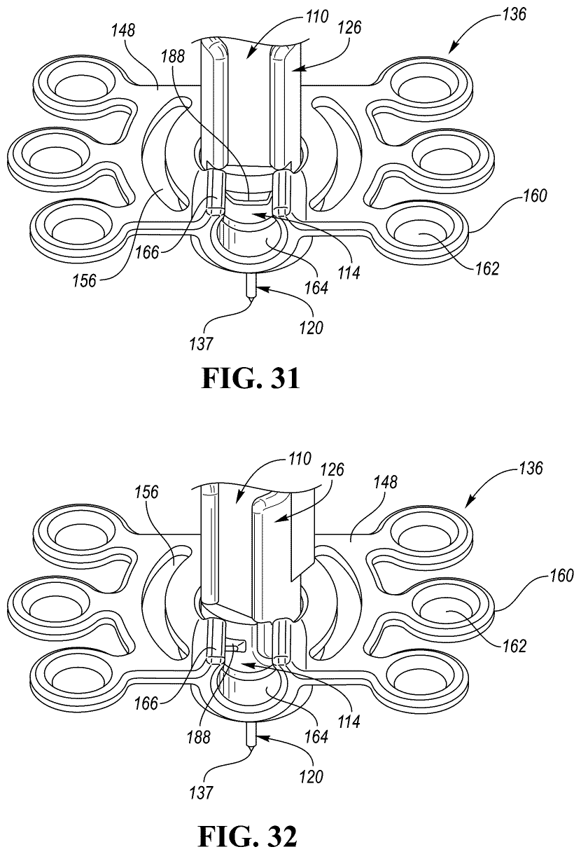

FIG. 31 is a top perspective view of the distal portion of the insertion assembly 102 and the implant 112 inserted into the guide plate 136 with the flange 188 of the outer housing 114 aligned with the transverse channel 164 of the guide plate 136 and the needle distal end 122 in position to have penetrated the dura D and into the venous blood-containing portion of the sagittal sinus S according to one or more embodiments. FIG. 32 is a top perspective view of the distal portion of the insertion assembly 102 and the implant assembly 104 with the flange 188 rotated approximately 45 degrees from the transverse channel 164 (such as by rotating the insertion assembly 102 and attached implant 112), illustrating initial locking of the implant 112 to the guide plate 136 by rotating the outer housing 114 so that the flange 188 engages an inner recess 154 of the central cylindrical portion 150 of the guide plate 136.

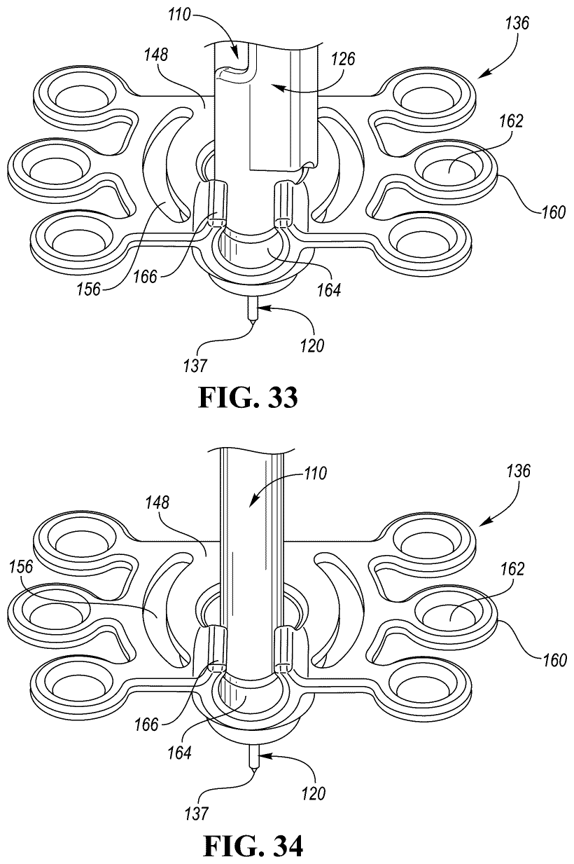

FIG. 33 is a top perspective view of the distal portion of the insertion assembly 102, wherein the flange 188 of the outer housing 114 has been rotated approximately 90 degrees from the transverse channel 164 (such as by rotating the insertion assembly 102 and attached implant 112) at which point the implant 112 is now fully locked to the guide plate 136. Advantageously, this locking of the implant 112 to the guide plate 136 is accomplished without putting any loads on the outer housing 114, the needle 120, or the stylet 124. At this point, as the stylet 124 has penetrated the dura D, the stylet 124 and the catheter sheath 126 can be removed from the insertion assembly 102. FIG. 34 is a top perspective view of the catheter 110 and the implant 112 locked to the guide plate 136 once the stylet 124 and the catheter sheath 126 have been removed according to one or more embodiments. In this position, the cutout 184 of the outer housing 114 is aligned with the transverse channel 164 of the guide plate 136.

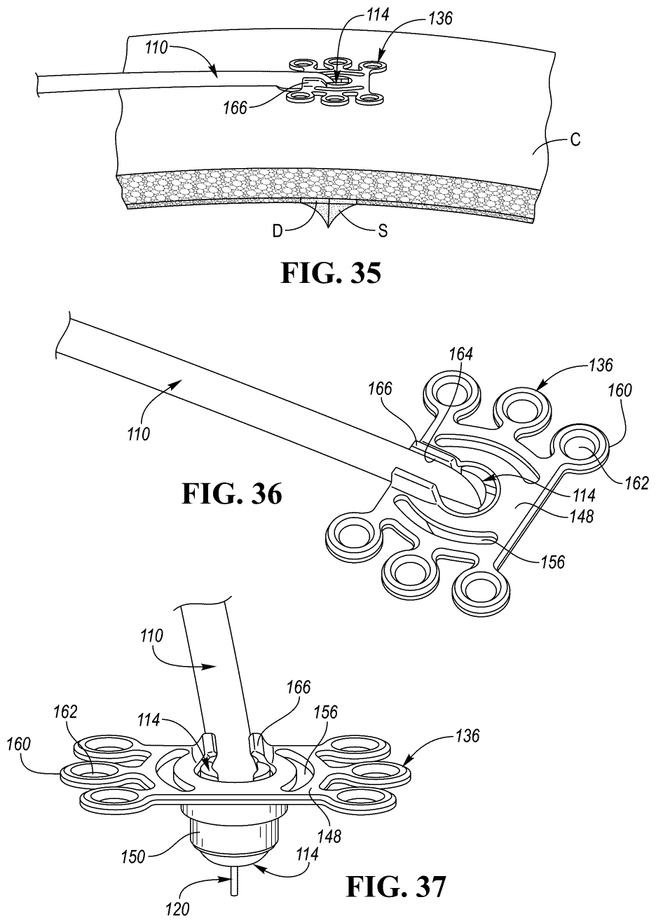

FIG. 35 is a top perspective view of the cranium C with the guide plate 136 and the implant 112 secured thereto after the stylet 124, the catheter sheath 126, the adapter lock 130, and catheter adapter 106 have been removed, wherein the catheter 110 is held within the transverse channel 164, such as within the arms 166, of the guide plate 136 thereby preventing unlocking of the catheter 110 from the guide plate 136 or movement of the outer housing 114 within the guide plate 136, according to one or more embodiments. FIG. 36 is a top perspective view of the guide plate 136 and secured catheter 110, and FIG. 37 is a front perspective view of the guide plate 136 and secured catheter 110.

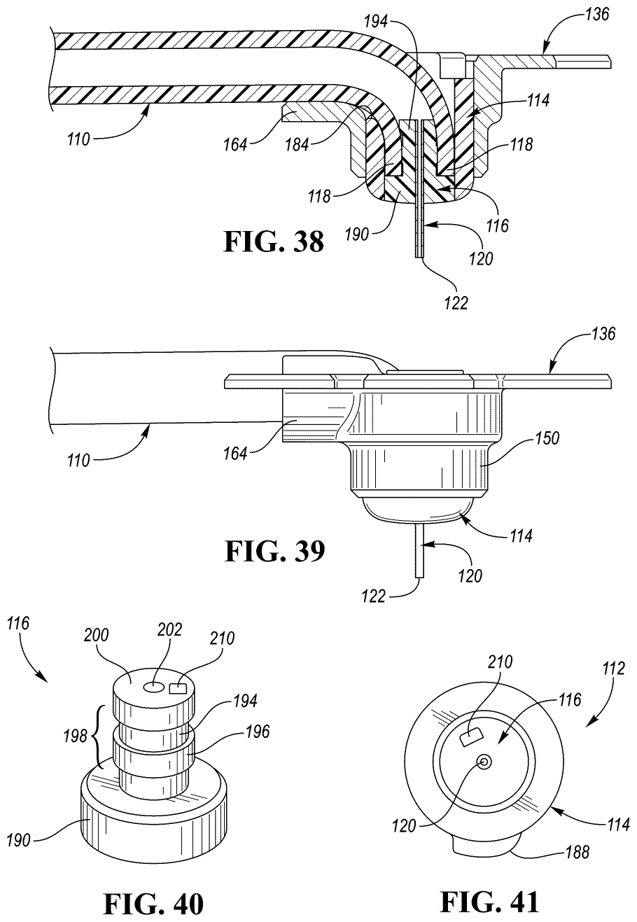

FIG. 38 is a cross-sectional view of the guide plate 136, the implant 112 with the distal end 118 of the catheter 110 secured to the upper portion 194 of the inner housing 116 and the outer housing 114 secured to the guide plate 136, and the catheter 110 held in the transverse channel 164 of the guide plate 136 after the stylet 124 and other components of the insertion assembly 102 have been removed. FIG. 39 is a front view of the guide plate 136, the received implant 112, and the secured catheter 110.

FIG. 40 is a perspective view of the inner housing 116 illustrating an embodiment wherein an integrated, surface-mounted sensor 210 is provided on the top end 200 of the inner housing 116 and configured for in vivo measurement of CSF pressure. FIG. 41 is a top view of the inner housing 116 disposed within the outer housing 114 illustrating an embodiment wherein an integrated, surface-mounted sensor 210 is provided on the base portion 190 of the inner housing 116 and configured to contact the dura D over the sagittal sinus S for in vivo measurement of sinus pressure.

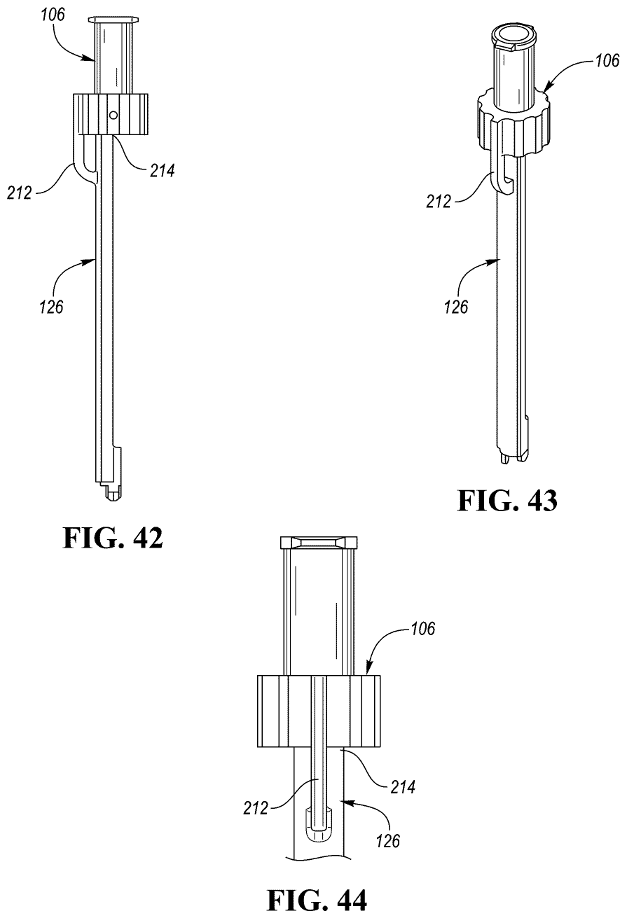

FIG. 42 is a side view of a catheter sheath 126 that eliminates the adapter lock 130 and replaces it with a breakaway member 212 joined between the catheter sheath 126 and the catheter adapter 106 according to one or more embodiments. FIG. 43 is a perspective view of the catheter sheath 126 of FIG. 42, and FIG. 44 is a cutaway, front view of the catheter sheath 126 of FIG. 42 illustrating a gap 214 between the catheter adapter 106 and the catheter sheath 126.

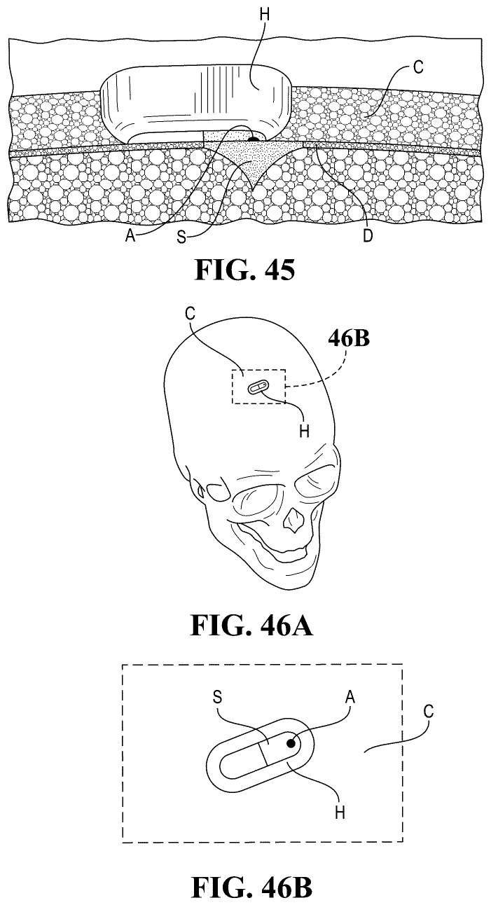

FIG. 45 shows the cranium C and the sagittal sinus S, and illustrates a first step in a surgical method utilizing the shunt system 100 according to one or more embodiments. The first step includes removing the cranial bone starting lateral of the sagittal sinus S down to the inner table, and then removing the inner table and progressing medially until the midline of the sagittal sinus S (located on the longitudinal axis of the sagittal sinus S) is exposed, forming a burr hole H or slot in the cranium C. FIG. 46 shows a top view of the burr hole H in the cranium C and the sagittal sinus S, and illustrates a continuation of the first surgical step according to one or more embodiments, including locating the longitudinal axial centerline or midline of the sagittal sinus S and marking the intended implant location with an aiming dot A. Exemplary, non-limiting dimensions of the burr hole H may be approximately 15 mm in length and approximately 7 mm in width, but other dimensions are fully contemplated.

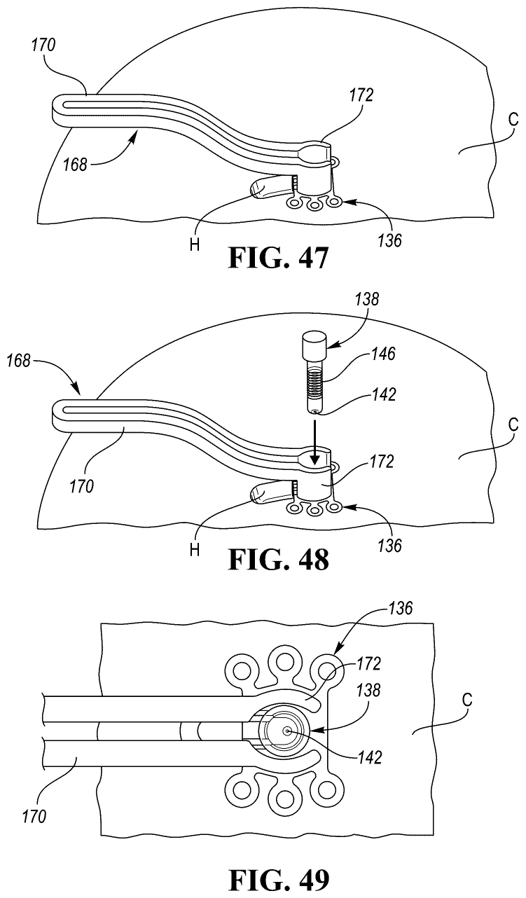

FIG. 47 shows a perspective view of the guide plate holder 168 and the guide plate 136, and illustrates a second step in the surgical method according to one or more embodiments, including placing the guide plate 136 into the burr hole H in the cranium C using the guide plate holder 168. Of course, it is also contemplated that the guide plate 136 may be placed in the burr hole H without use of the guide plate holder 168. FIG. 48 shows a perspective view of the depth gauge 138 prior to insertion into the guide plate holder 168 and the guide plate 136, and illustrates a continuation of the second surgical step according to one or more embodiments, including inserting the depth gauge 138 through the guide plate holder 168 and into the guide plate 136. FIG. 49 shows a top view of the guide plate holder 168 and the guide plate 136 with the depth gauge 138 inserted, and illustrates a third step in the surgical method according to one or more embodiments, including aligning a guidance marker 142 of the depth gauge 138 with the surgeon-placed aiming dot A indicating the longitudinal axial centerline or midline of the sagittal sinus S. As described above, the depth gauge 138 could also be used with the guide plate 136 in the absence of the guide plate holder 168.

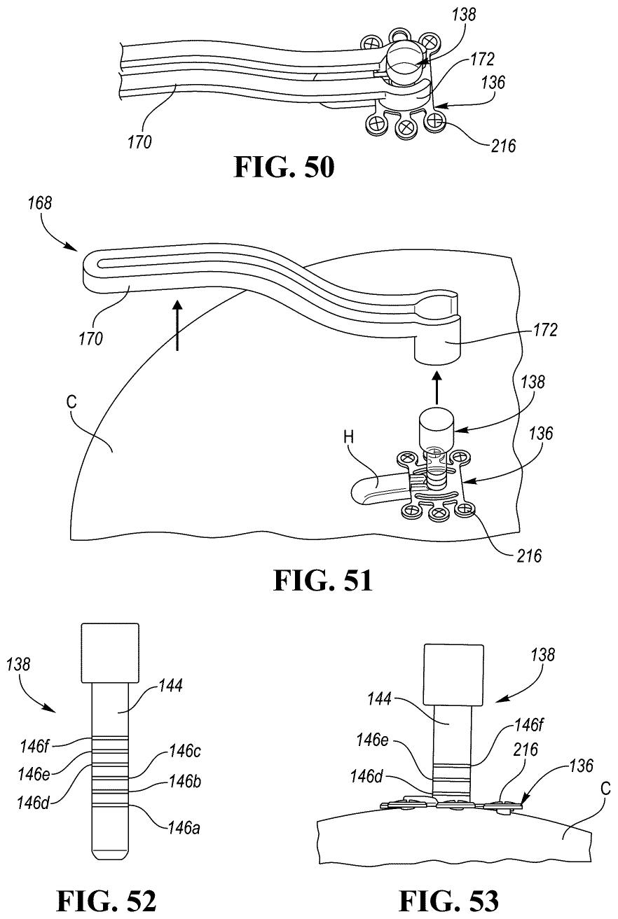

FIG. 50 shows a perspective view of the depth gauge 138 received in the guide plate 136, and illustrates a fourth step in the surgical method according to one or more embodiments, including securing the guide plate 136 to the cranium C using fasteners 216 such as, but not limited to, flush, low-profile CMF bone screws, through the tabs 160 and apertures 162 in the guide plate 136, ensuring that the guide plate 136 stays in a desired position with respect to the sagittal sinus S. FIG. 51 shows the guide plate holder 168 removed from the guide plate 136 with the depth gauge 138 remaining in the guide plate 136, and illustrates a continuation of the fourth surgical step according to one or more embodiments, including removing the guide plate holder 168 once the guide plate 136 is secured to the cranium C.

FIG. 52 is a front view of the depth gauge 138 having a plurality of depth markings 146 used to determine the thickness of the cranium C from the guide plate 136 to the dura D in order to select the desired shunt system 100 (i.e., insertion assembly 102 with the correct depth of the outer housing 114) for the measured cranium thickness at the desired implant location according to one or more embodiments. FIG. 53 is a side view of the depth gauge 138 inserted in the guide plate 136, and illustrates a fifth step in the surgical method according to one or more embodiments wherein the depth gauge 138 is used to determine the correct sizing of the insertion assembly 102 and the implant 112 (specifically, the outer housing 114). The depth markings 146 on the depth gauge 138 may include, but are not limited to, different shades, colors, patterns, or indicia to indicate the thickness of the cranium C. In one non-limiting example, the depth markings 146 can indicate the following:

| | Cranium Thickness | | |
|---|---|---|---|
| | (mm) | | |
| Size | Recess 1 (154) | Recess 2 (154) | Recess 3 (154) |
| RED (146a) | 5 | 5.5 | 6 |
| WHITE (146b) | 6.5 | 7 | 7.5 |
| BLUE (146c) | 8 | 8.5 | 9 |
| GREEN (146d) | 9.5 | 10 | 10.5 |
| ORANGE (146e) | 11 | 11.5 | 12 |
| BLACK (146f) | 12.5 | 13 | 13.5 |

Of course, the number and spacing of the depth markings 146 shown and described herein is merely exemplary, and other configurations are fully contemplated. In the chart above, exemplary distance spacing is provided for each of the inner recesses 154 of the guide plate 136 into which the flange 188 may be received (see, for example, FIGS. 26-27). Again, it is understood that the number and spacing of the inner recesses 154 is merely exemplary, and other configurations are also possible.

For this surgical step, the depth marking 146 closest to the top plate 148 of the guide plate 136 when the depth gauge 138 is inserted may indicate the depth of the outer housing 114 that should be selected for the implant 112. In one or more embodiments, the insertion assembly 102 may come preassembled with a particular depth of outer housing 114, and so the most appropriate insertion assembly 102 may be selected based solely on the depth measurement from the depth gauge 138. After this depth is measured, the depth gauge 138 may be removed from the guide plate 136.

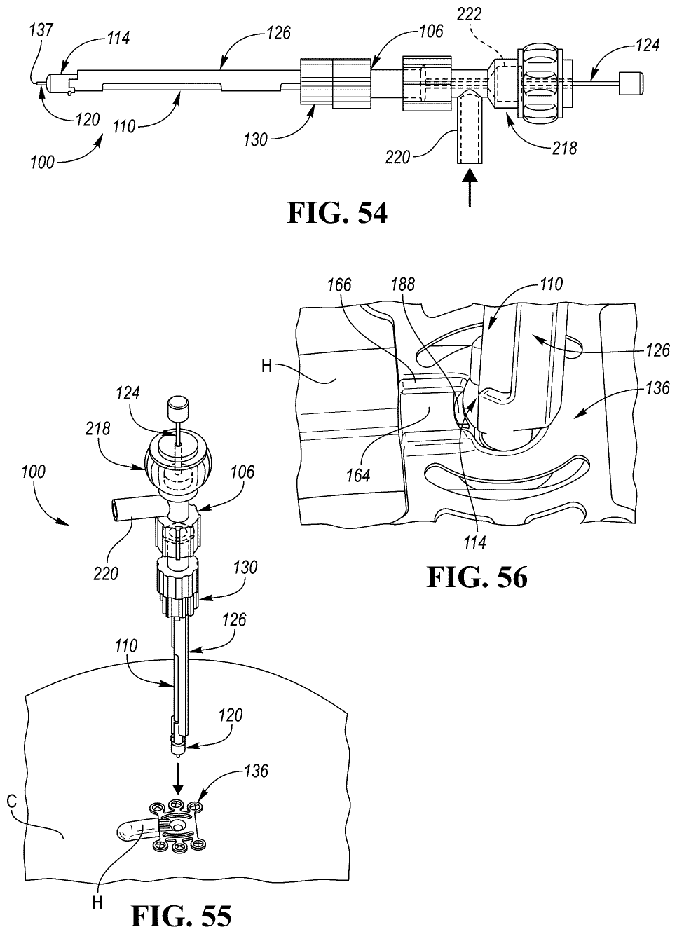

FIG. 54 is a side view of the insertion assembly 102 in an embodiment with a Tuohy Borst adapter 218 attached to the catheter adapter 106 and having a port 220 for saline, and illustrates a sixth step in the surgical method according to one or more embodiments wherein the outer housing 114 is selected as indicated by the depth measurement using the depth gauge 138 and the insertion assembly 102 is flushed with sterile saline. The Tuohy Borst adapter 218 (or other catheter adapter 106) may include a membrane 222 so that it is airtight and liquid tight, and is configured to receive the stylet 124 therethrough. As shown, the port 220 may extend at approximately 90 degrees and allows the surgeon to connect the insertion assembly 102 to a manometer (not shown) full of saline. The manometer may be held above the level of the surgery site in order to always be able to generate a higher pressure zone of the saline relative to the intracranial pressure (ICP) of the patient and to be assured that no reverse fluid flow or air-entrapment can occur. This is further done so that when the implant 112 is being inserted, the catheter 110 and the needle 120 are completely fluid filled, with no entrapped air, and such that there will always be a small amount of saline dripping out the needle distal end 122 during the surgical implantation.

FIG. 55 is a perspective view of the insertion assembly 102 and attached implant 112 prior to insertion into the guide plate 136, and illustrates a seventh step in the surgical method according to one or more embodiments wherein the properly sized, sterile, saline-filled insertion assembly 102 and implant 112 are inserted into the guide plate 136. FIG. 56 is an enlarged view of the insertion assembly 102 and implant 112 received in the guide plate 136 with the flange 188 of the outer housing 114 aligned with the transverse channel 164 of the guide plate 136, and illustrates a continuation of the seventh surgical step according to one or more embodiments wherein the insertion assembly 102 and implant 112 are inserted into the guide plate 136 with the outer housing flange 188 aligned with the transverse channel 164 of the guide plate 136.

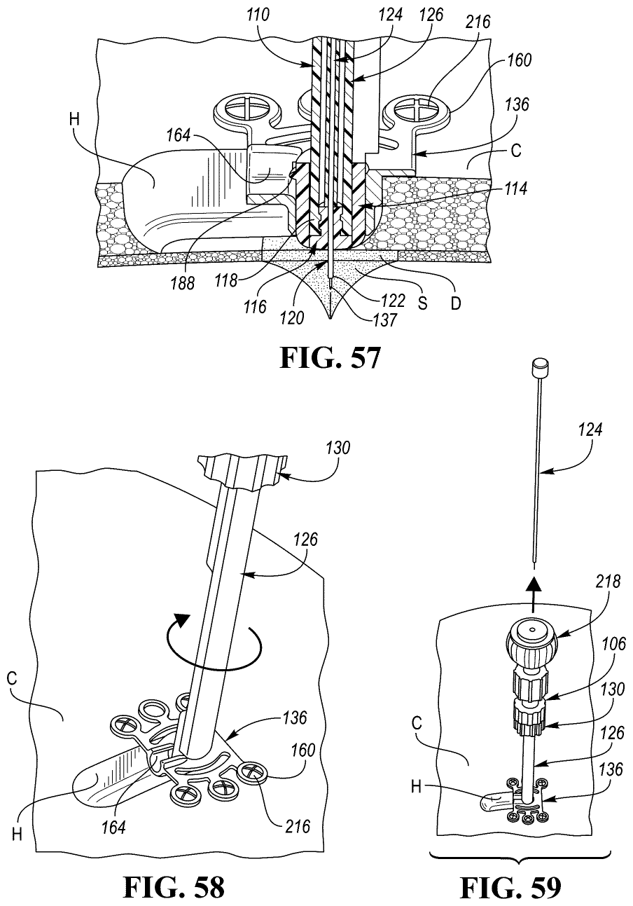

FIG. 57 is a cross-sectional view of the implant 112 received in the guide plate 136 with the needle distal end 122 in the sagittal sinus S, and illustrates an eighth step of the surgical method according to one or more embodiments wherein the insertion assembly 102 and implant 112 are inserted into the guide plate 136 until the outer housing 114 (i.e. bottom surface 180) contacts the bottom of the burr hole H and the stylet 124 penetrates the dura D into the sagittal sinus S. FIG. 58 is a perspective view of the insertion assembly 102 received in the guide plate 136, and illustrates a continuation of the eighth surgical step according to one or more embodiments, wherein the insertion assembly 102 is rotated approximately 90 degrees to secure the implant 112 to the guide plate 136 via engagement of the outer housing 114, as described above.

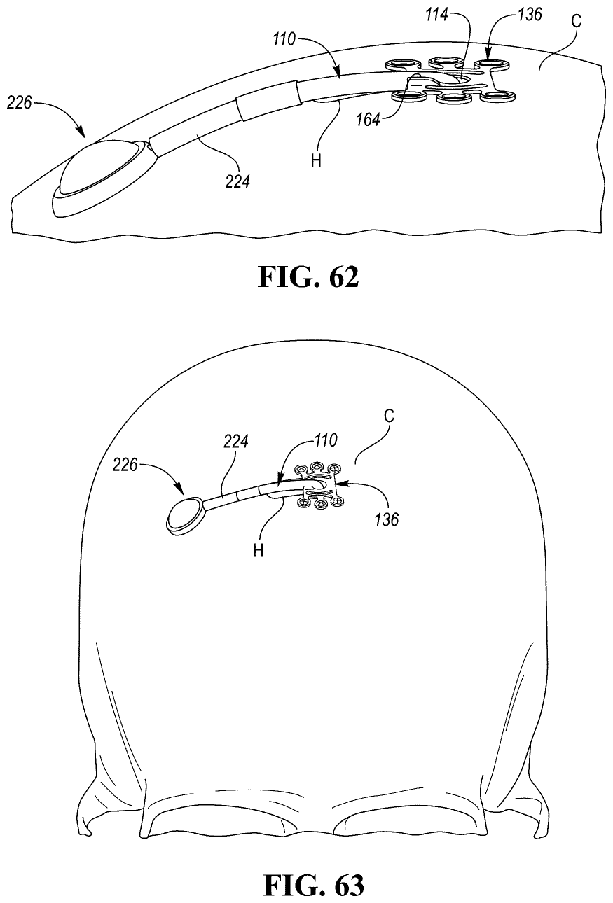

FIG. 59 is a perspective view of the insertion assembly 102, and illustrates a ninth step of the surgical method according to one or more embodiments including attaching a manometer (not shown) to the Tuohy Borst adapter 218 (or other catheter adapter 106) and filling the insertion assembly 102 with saline, then removing the stylet 124 and observing a drop in the manometer level showing patency by observing that the fluid level has dropped in the manometer once the implant 112 is positioned within the sagittal sinus S. The Tuohy Borst adapter 218 may be tightened during removal of the stylet 124 to prevent air inflow. In particular, once the stylet 124 is removed from the insertion assembly 102, the manometer will drain, confirming patency of the implant 112, until the pressure shown on the manometer is equal to the venous blood pressure in the patient's sagittal sinus S. Patency can also be confirmed by injecting a radio-opaque dye into the fluid and observing flow. Once patency is confirmed, then the shunt system 100 is ready to be connected to the proximal portion of a ventricular catheter 224 (see FIGS. 62-63) which has been previously placed in the patient's lateral ventricle or other areas containing CSF fluid around the brain.

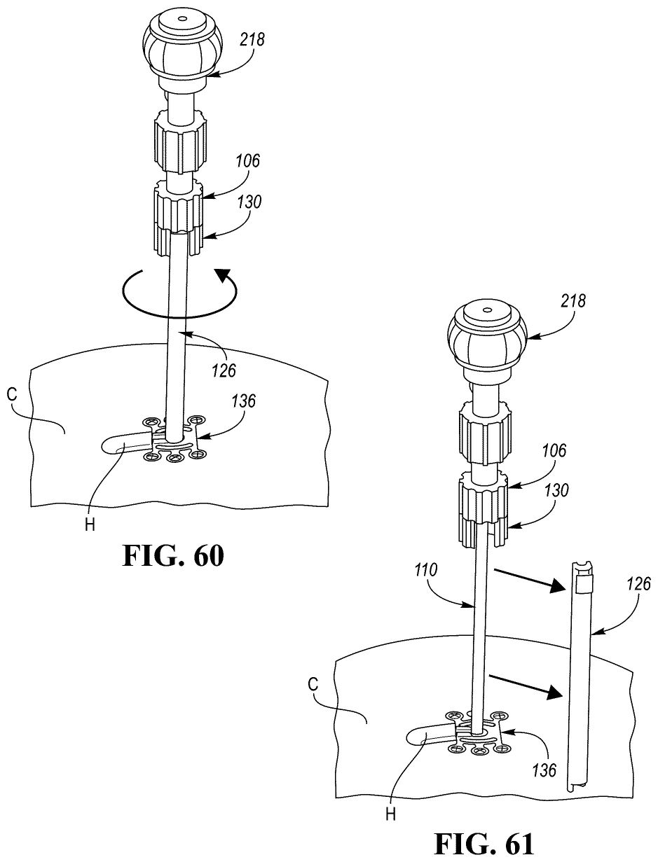

FIG. 60 is a perspective view of the insertion assembly 102, and illustrates a tenth step of the surgical method according to one or more embodiments, including removal of the catheter sheath 126 once patency has been demonstrated, such as by rotating the adapter lock 130 to an open position. FIG. 61 is a perspective view of the insertion assembly 102 and illustrates a continuation of the tenth surgical step, including removing the catheter sheath 126 from the insertion assembly 102. The adapter lock 130, the catheter adapter 106, and the Tuohy Borst adapter 218 can all also be removed at this point, and the catheter 110 clamped.

FIG. 62 is an enlarged view of the cranium C showing the guide plate 136, secured catheter 110, and proximal shunt components 226 including the ventricular catheter 224, and illustrates an eleventh step in the surgical method according to one or more embodiments, including bending the catheter 110 over and into the transverse channel 164 in the guide plate 136 and connecting the catheter 110 to the ventricular catheter 224 and other proximal shunt components 226 for draining CSF into the venous system (sagittal sinus S). FIG. 63 is a perspective view of the cranium C illustrating the guide plate 136, the secured catheter 110, and, for example, connected proximal shunt components 226 including a one way valve, the ventricular catheter 224 coming from the ventricle, and a burr hole reservoir.

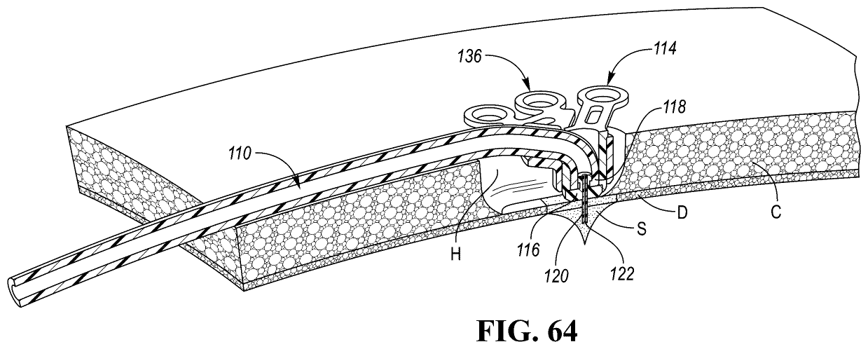

FIGS. 64-92 illustrate another embodiment of the shunt system 100. It is understood that all of the components, features, and functions shown and described above with reference to FIGS. 1-63 may also be applicable to the embodiment of FIGS. 64-92, and vice versa.

As above, the implant 112 may include an outer housing 114 and an inner housing 116, wherein a distal end 118 of a catheter 110 is arranged to be secured to the inner housing 116. A needle 120 may be received in the inner housing 116 at the distal end 118 of the catheter 110, the needle 120 having a distal end 122, wherein a penetrating stylet 124 is configured to protrude past the needle distal end 122 to facilitate insertion through the superior dura surface D of the superior sagittal sinus S and into the venous bloodstream. A catheter sheath 126 may at least partially surround a body 128 of the catheter 110 between the proximal end 108 and the distal end 118, and an adapter lock 130 may be coupled to the catheter adapter 106 to secure the catheter adapter 106 onto the catheter 110. The insertion assembly 102 and implant 112 are configured to be received in a guide plate 136. The shunt system 100 is provided to the surgeon in a pre-assembled, sterile package. Also as above, the inner housing 116 and the hollow needle 120 and the guide plate 136 may be common (of standard size) for each implant assembly 104, with the only variable component being the outer housing 114, namely the depth dimension thereof. The insertion assembly 102 and implant 112 as shown in FIGS. 64-92 may be provided to the surgeon, fully assembled (including the stylet 124).

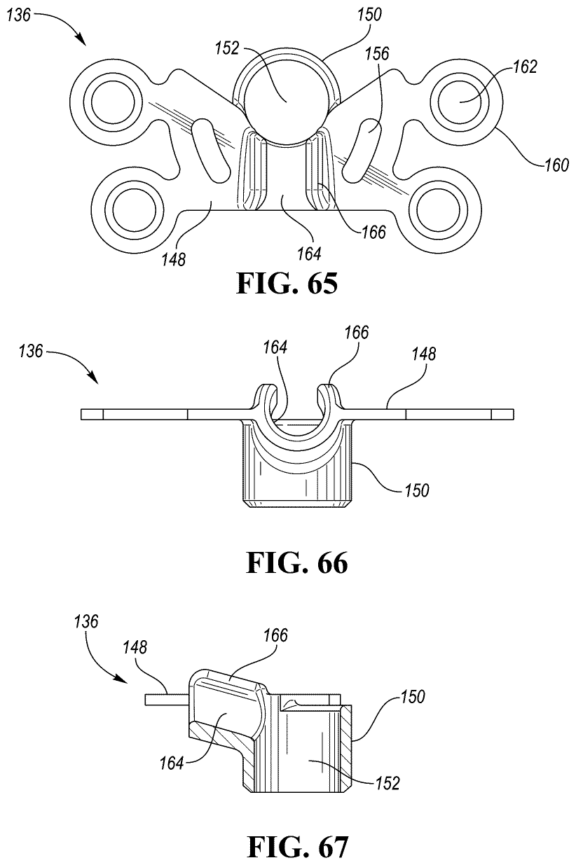

FIG. 64 shows a perspective view of the cranium C, the dura D, and the sagittal sinus S receiving an implant 112, connected catheter 110, and guide plate 136, where FIGS. 65-67 illustrate views of the guide plate 136. With reference to FIGS. 64-67, the guide plate 136 may include a generally planar top plate 148 with a central cylindrical portion 150 extending downwardly therefrom with a central lumen 152 therethrough. The top plate 148 may have slots 156 on either side of a top end 158 of the central cylindrical portion 150, and a plurality of tabs 160 with apertures 162 for receiving fasteners 216 (e.g. screws) to secure the guide plate 136 to the cranium C. Although four tabs 160 and corresponding apertures 162 are illustrated, it is understood that other configurations are also contemplated. A transverse channel 164 is formed in communication with the central lumen 152 and may have opposed arms 166 extending upwardly above the top plate 148. As described above, a guide plate holder 168 may include a distal head portion 172 with two spaced flanges 174 extending downwardly below the handle portion 170 at a distal end 176 of the guide plate holder 168, wherein the flanges 174 are configured to engage the corresponding slots 156 in the guide plate 136. The guide plate 136 is secured to the cranium C with fasteners 216 (e.g. flush, low-profile CMF bone screws) through the tabs 160 in the guide plate 136 as described above, ensuring that the guide plate 136 stays in the desired position with respect to the sagittal sinus S.

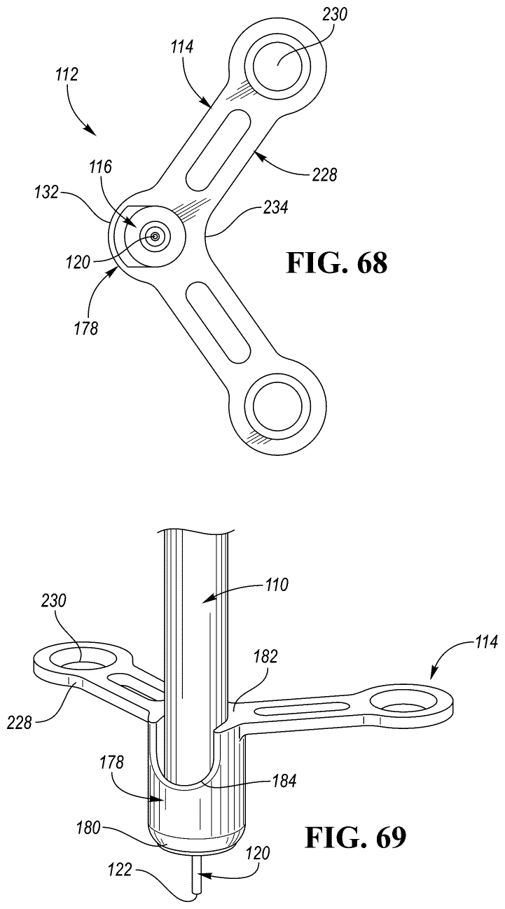

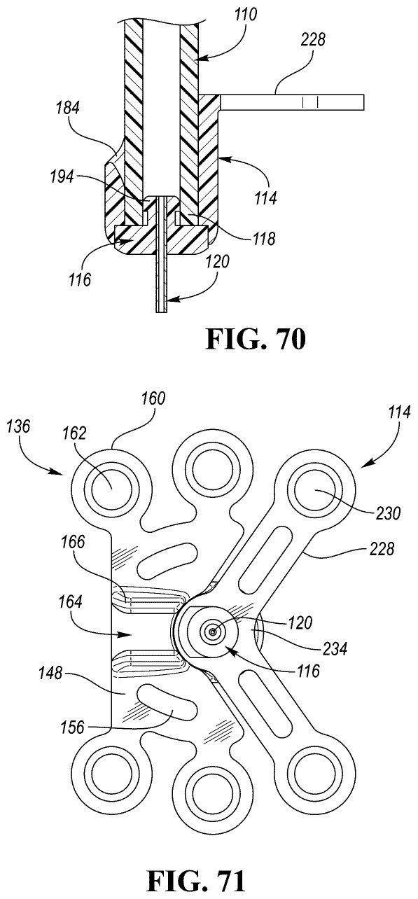

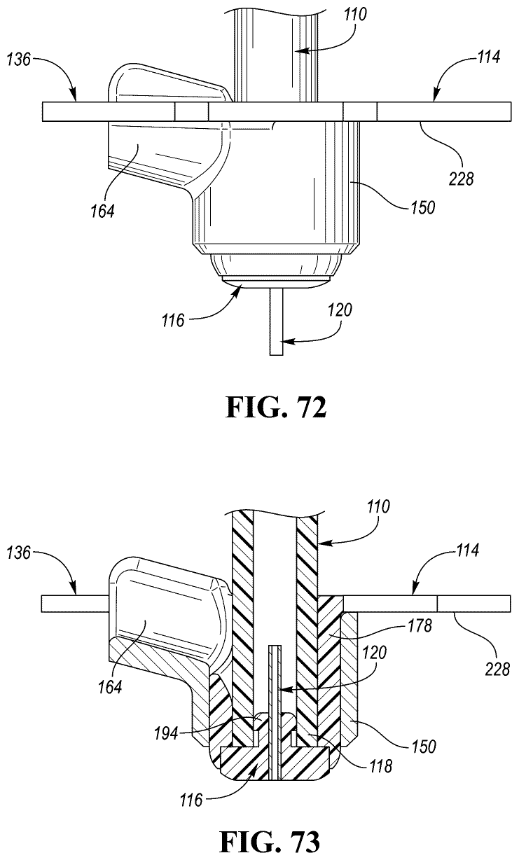

FIGS. 64 and 68-72 illustrate views of the outer housing 114 of the implant 112. FIG. 68 is a top view of an assembled implant 112, illustrating the outer housing 114 and the inner housing 116. FIGS. 69-70 are views of the assembled implant 112 with a distal end 118 of the catheter 110 attached to the inner housing 116. FIG. 71 is a top view of the implant assembly 104 including the implant 112 and the guide plate 136, and FIG. 72 is a side view of the implant assembly 104 with the catheter 110 attached to the inner housing 116.

The outer housing 114 may have a generally cylindrical body 178 with a rounded bottom surface 180. In one or more embodiments, the shape of the bottom surface 180 may be complementary to a 4 mm burr, for example. As illustrated in FIGS. 64 and 71, in this embodiment the body 178 of the outer housing 114 is configured to be received in the central lumen 152 of the guide plate 136 and attached directly to the cranium C, rather than attached to the guide plate 136. As such, in this embodiment, the flange 188 and inner recesses 154 may be absent. Receiving the outer housing 114 in the guide plate 136 allows these components to be co-located, with the guide plate 136 positioned over the intended target (e.g. the longitudinal midline of the sagittal sinus S). The top surface 182 of the outer housing 114 may be asymmetrical, with a rounded cutout 184 formed downwardly from the top surface 182, and a plurality of arms 228 (e.g. a pair of arms) extending outwardly from the outer housing 114 and terminating in apertures 230 for receiving fasteners 216 (e.g. screws) to secure the outer housing 114 to the cranium C. In one or more embodiments, the cutout 184 may be formed on a front side 232 of the body 178 and the arms 228 may extend outwardly generally from a rear side 234 of the body 178. In one or more embodiments, when the outer housing 114 is received in the guide plate 136, the cutout 184 may be configured to be aligned with the transverse channel 164, and the arms 228 may be configured to lie adjacent to the tabs 160 of the guide plate 136 (see FIG. 73). As above, the outer housing 114 may be provided in a plurality of different depths (for example, but not limited to, 5.5 mm, 7.0 mm, 8.5 mm, 10.0 mm, 11.5 mm, and 13.0 mm), the most suitable of which may be selected depending on the thickness of the patient's cranium C at the desired implant location as described herein.

FIGS. 68-72 illustrate views of the assembled implant 112 with the inner housing 116 received in the outer housing 114, wherein the inner housing 116 may be press fit or bonded to the outer housing 114. As above, the inner housing 116 may include a cylindrical base portion 190 which may be arranged to abut or engage an interior bottom surface 192 of the outer housing 114. An upper portion 194 of the inner housing 116 has a smaller diameter than the base portion 190, and includes circumferential protrusions 196, such as in the middle region 198 and the top end 200 of the upper portion 194. The distal end 118 of the catheter 110 is configured to be received on the upper portion 194 of the inner housing 116, such as by stretching it over the protrusions 196, adhesively bonding the catheter 110 to the inner housing 116, and/or attaching the catheter 110 to the inner housing 116 with suture, for example. The inner housing 116 has a lumen 202 therethrough with a needle 120 having a distal end 122 extending downwardly from the base portion 190 and past the bottom surface 180 of the outer housing 114 when the outer housing 114 and the inner housing 116 are assembled together.

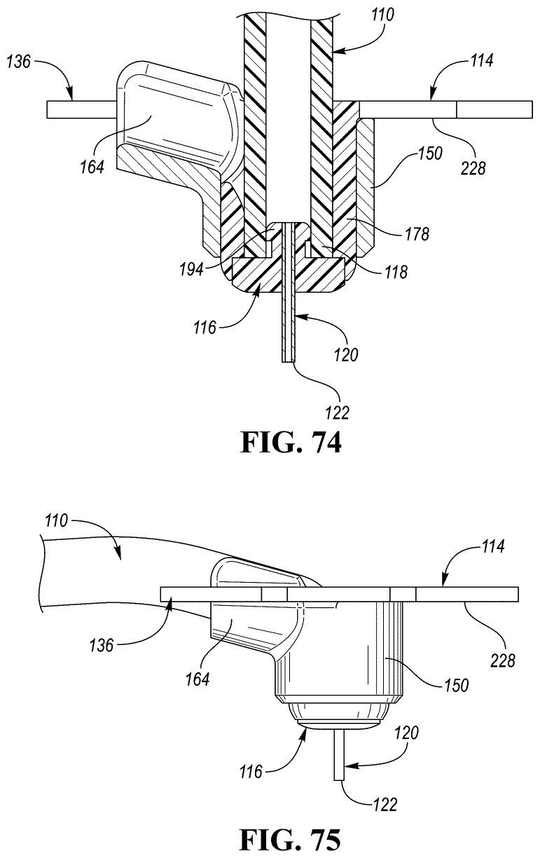

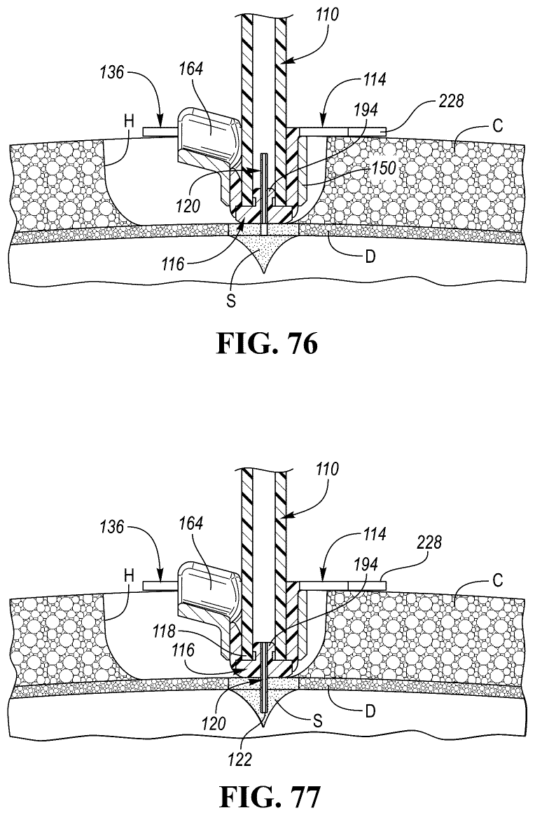

FIGS. 73-74 are cross-sectional views of the assembled implant 112, catheter 110, and guide plate 136 illustrating an embodiment wherein the needle 120 and its distal end 122 may be retractable (FIG. 73) and extendable (FIG. 74) within and through the inner housing 116. Similarly, FIGS. 76 and 77 are cross-sectional views of the assembled implant 112, catheter 110 and guide plate 136 within the created burr hole H in the cranium C and in relationship to the dura D and sagittal sinus S, both with the needle 120 retracted (FIG. 76) and extended (FIG. 77) with respect to the inner housing 116. This embodiment provides additional protection against inadvertent bending of the needle distal end 122 as the outer housing 114 is being received in the guide plate 136 and positioned on the dura D by keeping the needle distal end 122 retracted within the implant 112 during this process. Once the outer housing 114 is positioned as desired and the outer housing 114 and the guide plate 136 (or just the guide plate 136, see the embodiments of FIGS. 1-63) are attached to the cranium C, then the needle distal end 122 may be extended, such as via advancement of the stylet 124 and its engagement with the inner housing 116.

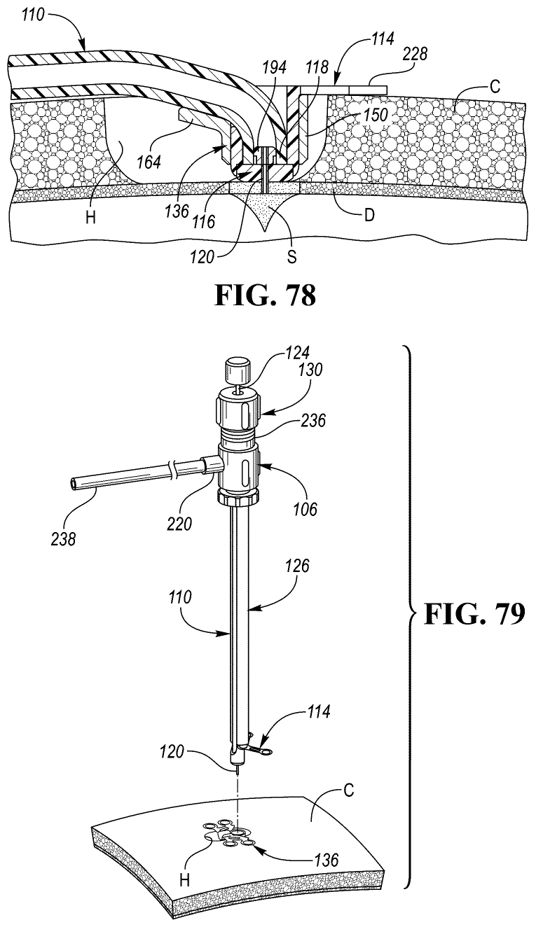

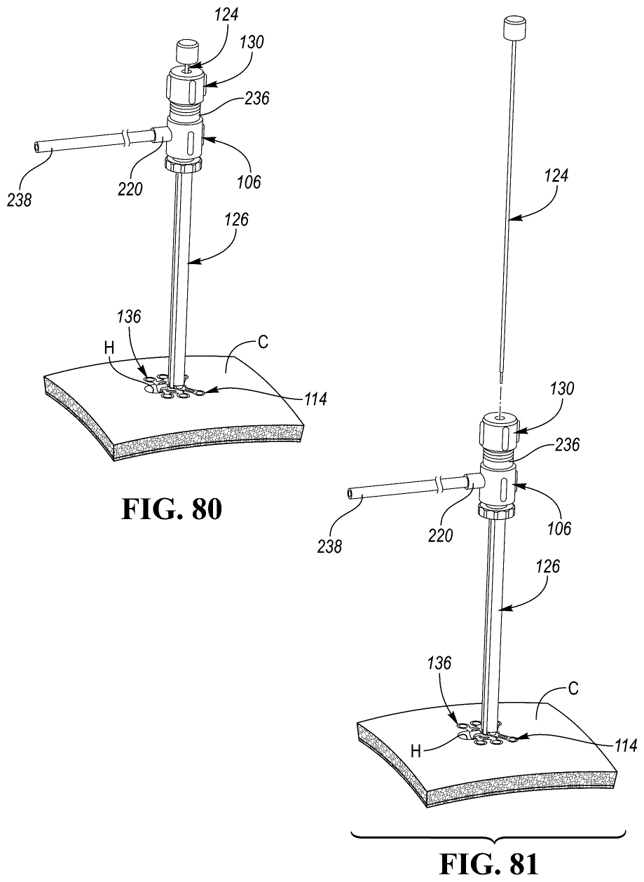

FIG. 78 illustrates the outer housing 114 received in the guide plate 136, the needle distal end 122 extended from the inner housing 116, and the catheter 110 received in the transverse channel 164 of the guide plate 136, preventing unlocking of the implant 112 from the guide plate 136 or movement of the outer housing 114 within the guide plate 136. In FIGS. 79-80, views of the insertion assembly 102 and implant 112 are illustrated prior to engagement with the guide plate 136 positioned on the cranium C (FIG. 79), and then after engagement of the insertion assembly 102 and implant 112 with the guide plate 136, with the outer housing 114 received in the guide plate 136 and secured to the cranium C (FIG. 80). In one or more embodiments, the outer housing 114 is inserted in the guide plate 136 with the inner housing 116, the needle 120, and the catheter 110 already assembled thereto. Once the needle distal end 122 is inserted into the dura D, the outer housing 114 may be fastened to the cranium C. As described above, in the insertion assembly 102, an internal, solid-tipped, sharp stylet 124 fits inside the catheter 110 and through the needle 120 so as to extend past the distal end 122. Engagement of the insertion assembly 102 with the guide plate 136 and removal of the stylet 124 from the catheter 110 is illustrated in FIG. 81, such as by loosening the adapter lock 130.

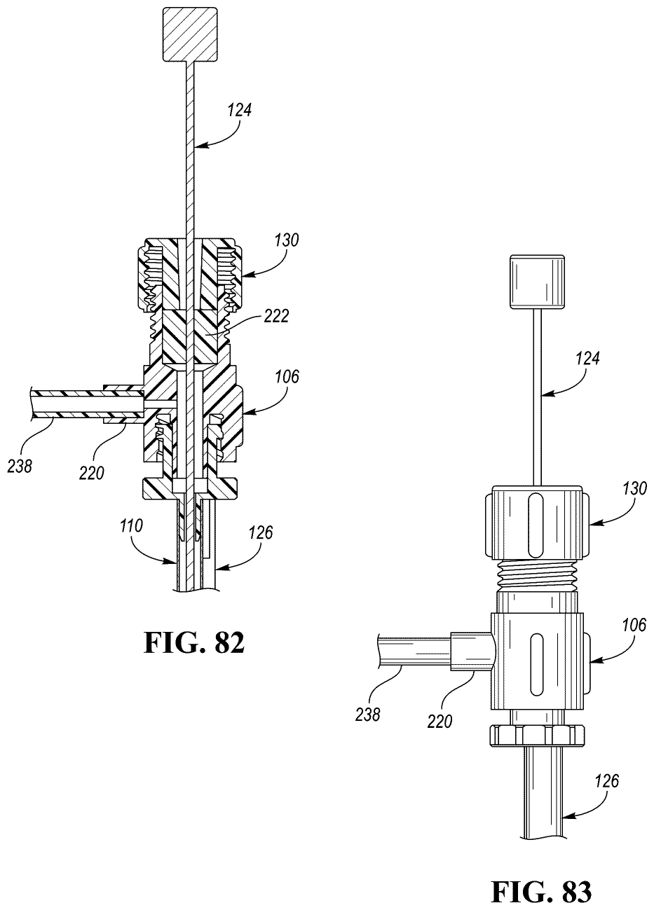

FIGS. 82-83 illustrate a proximal portion of the insertion assembly 102 including a catheter adapter 106 having a distal portion connected to the catheter 110, a side port 220 which may extend at approximately 90 degrees and be configured to receive saline-filled tubing 238, and a proximal portion for receiving an adapter lock 130, the catheter adapter 106 including a stylet seal or membrane 222, and a stylet 124 received through the adapter lock 130, the membrane 222, and the catheter adapter 106 to extend into the catheter 110, with a catheter sheath 126 at least partially encircling the catheter 110. The membrane 222 may comprise a piece of urethane or silicone rubber which is airtight and liquid tight that the stylet 124 is inserted through, and the adapter lock 130 may be used to create resistance to hold the stylet 124 in position and prevent any fluid leakage.

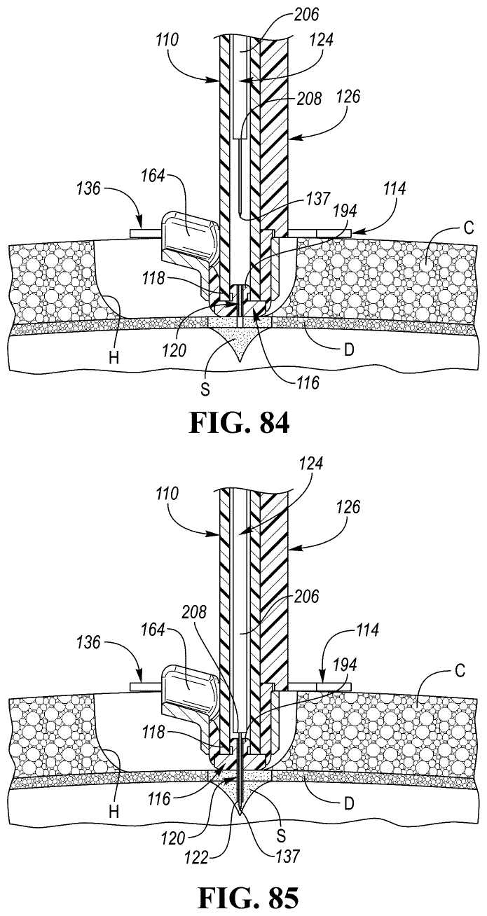

FIG. 84 is a cross-sectional view of the assembled implant 112 and guide plate 136 with the stylet 124 retracted in the catheter 110 proximal to the needle distal end 122, and FIG. 85 is a cross-sectional view of the assembled implant 112 and guide plate 136 with the stylet 124 advanced in the catheter 110 to extend past the needle distal end 122. As best shown in FIGS. 84-85, there is an internal integrated depth stop for the stylet 124. When the stylet end portion 208 abuts the top end 200 of the inner housing 116, the distal placement of the stylet 124 is halted such that the amount of exposed sharpened stylet tip 137 coming through the needle distal end 122 is limited to a desired amount of protrusion, typically less than 1 mm, for example. FIG. 85 illustrates the implant assembly 104 after the needle distal end 122 and the stylet tip 137 have penetrated the dura D, wherein the implant 112 (outer housing 114 and/or the inner housing 116) may be engaging the dura D and the guide plate 136 is secured to the cranium C.

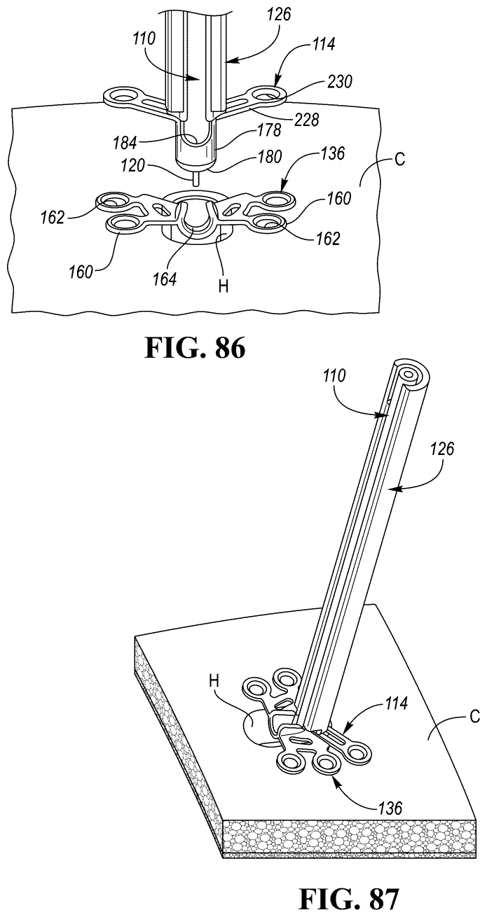

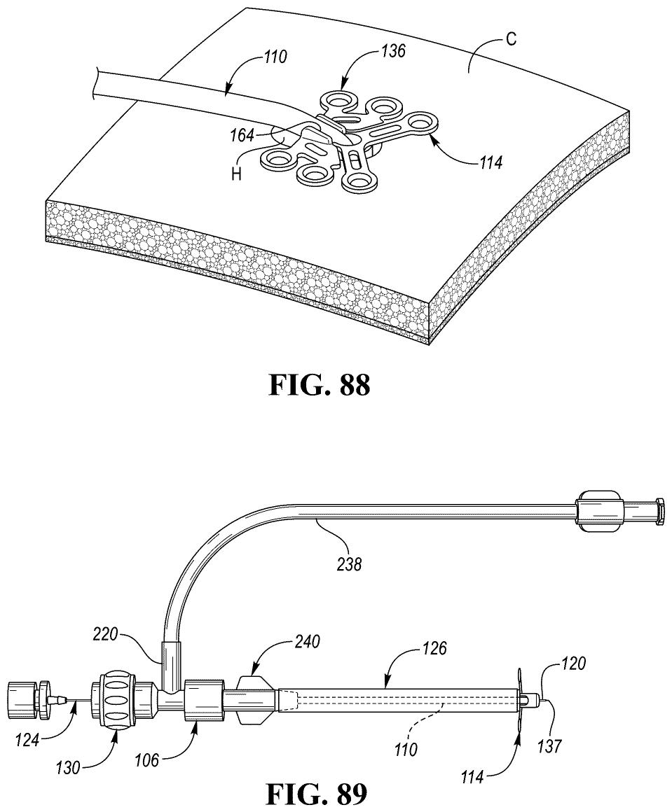

FIG. 86 is a perspective view of the insertion assembly 102 and implant 112 prior to insertion into the guide plate 136, and FIG. 87 is a perspective view of the insertion assembly 102 and implant 112 after insertion into the guide plate 136. Once patency has been demonstrated, the catheter sheath 126 may be removed, such as by rotating the adapter lock 130. FIG. 88 is a perspective view of the implant assembly 104 with the guide plate 136 and outer housing 114 secured to the cranium C and the catheter 110 received in the transverse channel 164 of the guide plate 136, after which the catheter 110 may be connected to proximal shunt components 226 for draining CSF into the venous system (sagittal sinus S).

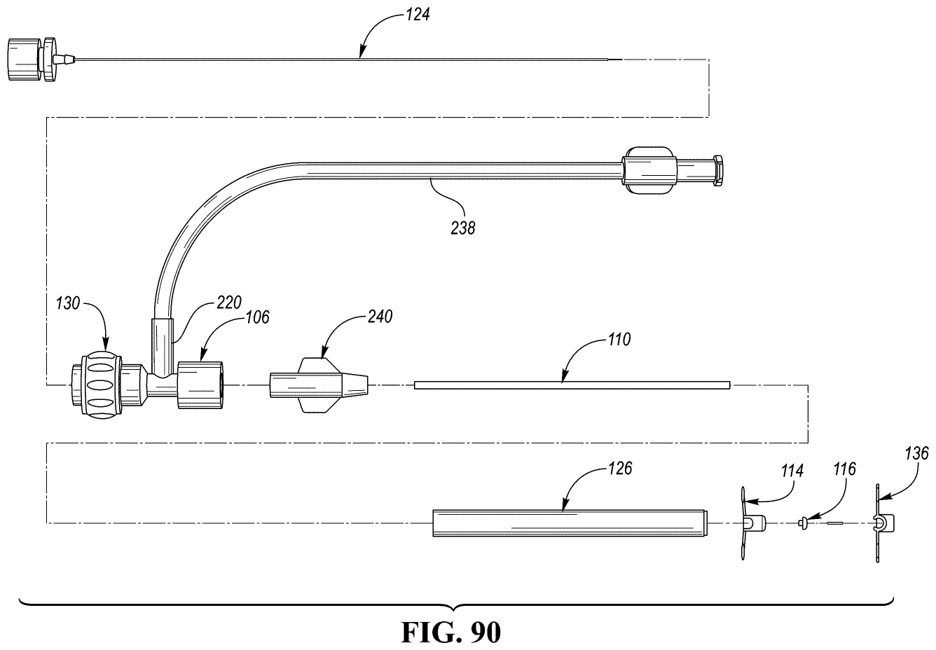

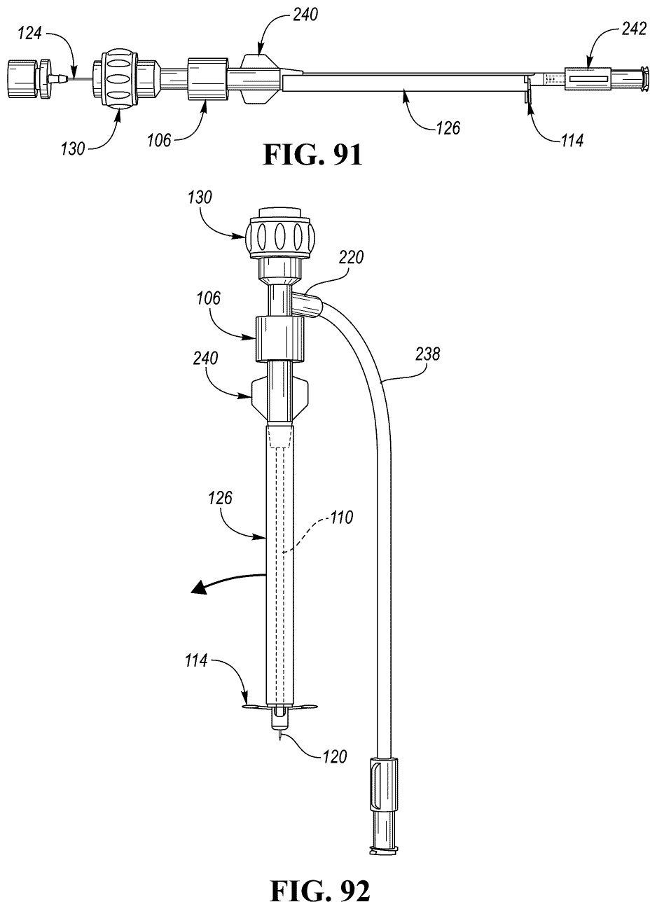

FIG. 89 is a side view of components of the insertion assembly 102 and implant 112 and FIG. 90 is an exploded view of components of the insertion assembly 102 and the implant assembly 104. FIG. 90 illustrates the catheter adapter 106 with the adapter lock 130, saline-filled tubing 238 received on the side port 220 of the catheter adapter 106 and including a distal connector 240 (Luer lock) for interfacing between the catheter adapter 106 and the proximal end 108 of the catheter 110. FIG. 91 is a side view of the insertion assembly 102 with a removable protective cover 242 for the needle distal end 122, such as for use during packaging and transport. FIG. 92 is a perspective view of components of the insertion assembly 102 and implant 112 illustrating removal of the catheter sheath 126.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A shunt system configured for draining excess cerebrospinal fluid (CSF) into a venous system of a patient via a burr hole created in a cranium at a desired implant location, the shunt system comprising:
an insertion assembly including a catheter having a proximal end and a distal end, a catheter sheath at least partially surrounding a body of the catheter between the catheter proximal end and the catheter distal end, and a stylet received in the catheter and having a tip;
an implant coupled to the insertion assembly and disposed at the catheter distal end, the implant including an outer housing, an inner housing received within the outer housing, and a hollow needle extending from the inner housing and past a bottom surface of the outer housing, the needle having a distal end; and
a guide plate configured to receive the implant and be secured to the cranium over the burr hole, wherein the stylet tip protrudes past the needle distal end such that the needle distal end and the stylet tip are configured to penetrate a dura of a sagittal sinus exposed by the burr hole,
wherein the stylet and the catheter sheath are removable from the catheter such that the catheter is connectable to proximal shunt components for draining excess CSF into the venous system of the patient.

2. The shunt system of claim 1, wherein the guide plate is configured to engage the outer housing to secure the implant to the guide plate.

3. The shunt system of claim 1, wherein the catheter distal end is connected to the inner housing.

4. The shunt system of claim 1, wherein the insertion assembly, the implant, and the guide plate are provided in a pre-assembled, sterile package.

5. The shunt system of claim 1, wherein the inner housing, the needle, and the guide plate each have a standard size, and wherein the outer housing is provided in different depths to accommodate for a specific thickness of the cranium of the patient at a desired implant location.

6. The shunt system of claim 1, further comprising a depth gauge for aligning the guide plate with the desired implant location, wherein the depth gauge is configured to be removably received in the guide plate.

7. The shunt system of claim 6, wherein the depth gauge has a distal end with a guidance marker configured for aligning with a surgeon-placed aiming dot on the sagittal sinus, wherein a body of the depth gauge includes depth markings for determining a distance from the guide plate to the dura for selecting a suitable depth of the outer housing.

8. The shunt system of claim 1, wherein the guide plate includes a generally planar top plate with a central cylindrical portion extending downwardly therefrom having a central lumen therethrough for receiving the outer housing.

9. The shunt system of claim 8, wherein the outer housing includes a flange extending outwardly therefrom, wherein the central cylindrical portion includes one or more inner recesses configured to engage the flange and secure the outer housing to the guide plate.

10. The shunt system of claim 1, wherein the guide plate includes a plurality of tabs with apertures for receiving fasteners to secure the guide plate to the cranium.

11. The shunt system of claim 1, wherein the guide plate includes a transverse channel for receiving and securing the catheter.

12. The shunt system of claim 11, wherein the outer housing has a generally cylindrical body with a rounded bottom surface and a top surface having a cutout formed downwardly therefrom, wherein the cutout is configured to be aligned with the transverse channel for receiving the catheter.

13. The shunt system of claim 1, wherein the outer housing includes a plurality of arms terminating in apertures for receiving fasteners to secure the outer housing to the cranium.

14. The shunt system of claim 1, further comprising a guide plate holder configured to engage the guide plate to aid in placement of the guide plate during securing of the guide plate to the cranium.

15. The shunt system of claim 1, wherein the inner housing includes a base portion and an upper portion, the upper portion having a smaller diameter than the base portion, wherein the catheter distal end is received on the upper portion.

16. The shunt system of claim 15, wherein the upper portion includes circumferential protrusions.

17. The shunt system of claim 1, wherein the stylet includes a body having a larger diameter than an end portion thereof, the body configured to abut a top end of the inner housing to limit an amount of the tip protruding past the needle distal end.

18. The shunt system of claim 1, further comprising a catheter adapter disposed at the catheter proximal end.

19. The shunt system of claim 18, further comprising an adapter lock disposed on a distal portion of the catheter adapter and a proximal portion of the catheter sheath to secure the catheter adapter onto the catheter.

20. The shunt system of claim 1, wherein the inner housing includes a surface-mounted sensor.

21. The shunt system of claim 20, wherein the sensor is provided on a top end of the inner housing and is configured for in vivo measurement of CSF pressure.

22. The shunt system of claim 20, wherein the sensor is provided on a base portion of the inner housing and is configured to contact the dura for in vivo measurement of sinus pressure.

23. The shunt system of claim 1, wherein the needle is retractable and extendable with respect to the inner housing.

24. The shunt system of claim 23, wherein engagement of the stylet with the inner housing causes the needle to be extended from the inner housing.

25. The shunt system of claim 1, further comprising a removable cover for the needle distal end.

26. A shunt system configured for draining excess cerebrospinal fluid (CSF) into a venous system of a patient via a burr hole created in a cranium at a desired implant location, the shunt system comprising:
an insertion assembly including a catheter having a proximal end and a distal end, a catheter sheath at least partially surrounding a body of the catheter between the catheter proximal end and the catheter distal end, and a stylet received in the catheter and having a tip;
an implant coupled to the insertion assembly and disposed at the catheter distal end, the implant including an outer housing including a flange extending outwardly therefrom, an inner housing received within the outer housing, and a hollow needle extending from the inner housing and past a bottom surface of the outer housing, the needle having a distal end; and
a guide plate configured to receive the implant and be secured to the cranium over the burr hole, the guide plate including one or more inner recesses configured to engage the flange to secure the outer housing to the guide plate, wherein the stylet tip protrudes past the needle distal end such that the needle distal end and the stylet tip are configured to penetrate a dura of a sagittal sinus exposed by the burr hole,
wherein the stylet and the catheter sheath are removable from the catheter such that the catheter is connectable to proximal shunt components for draining excess CSF into the venous system of the patient.

27. A shunt system configured for draining excess cerebrospinal fluid (CSF) into a venous system of a patient via a burr hole created in a cranium at a desired implant location, the shunt system comprising:
an insertion assembly including a catheter having a proximal end and a distal end, a catheter sheath at least partially surrounding a body of the catheter between the catheter proximal end and the catheter distal end, and a stylet received in the catheter and having a tip;
an implant coupled to the insertion assembly and disposed at the catheter distal end, the implant including an outer housing configured to be secured directly to the cranium, an inner housing received within the outer housing, and a hollow needle extending from the inner housing and past a bottom surface of the outer housing, the needle having a distal end; and
a guide plate configured to receive the implant and be secured to the cranium over the burr hole, wherein the stylet tip protrudes past the needle distal end such that the needle distal end and the stylet tip are configured to penetrate a dura of a sagittal sinus exposed by the burr hole,
wherein the stylet and the catheter sheath are removable from the catheter such that the catheter is connectable to proximal shunt components for draining excess CSF into the venous system of the patient.

28. A method for draining excess cerebrospinal fluid (CSF) into a venous system of a patient via a burr hole created in a cranium at a desired implant location, the method comprising:
providing an insertion assembly including a catheter having a proximal end and a distal end, a catheter sheath at least partially surrounding a body of the catheter between the catheter proximal end and the catheter distal end, and a stylet received in the catheter and having a tip;
providing an implant coupled to the insertion assembly and disposed at the catheter distal end, the implant including an outer housing, an inner housing received within the outer housing, and a hollow needle extending from the inner housing and past a bottom surface of the outer housing, the needle having a distal end, wherein the stylet tip protrudes past the needle distal end;
receiving the implant in a guide plate;
securing the guide plate to the cranium over the burr hole;
penetrating a dura of a sagittal sinus exposed by the burr hole with the needle distal end and the stylet tip;
removing the stylet and the catheter sheath from the catheter; and
connecting the catheter to proximal shunt components for draining excess CSF into the venous system of the patient.

29. The method of claim 28, further comprising marking the desired implant location on the sagittal sinus with an aiming dot.

30. The method of claim 29, further comprising inserting a depth gauge into the guide plate and aligning a guidance marker of the depth gauge with the aiming dot.

31. The method of claim 30, further comprising using depth markings on the depth gauge to select the outer housing with a suitable depth based on a thickness of the cranium at the desired implant location.

32. The method of claim 28, further comprising placing the guide plate over the burr hole using a guide plate holder.

33. The method of claim 28, further comprising rotating the insertion assembly to secure the implant to the guide plate.

34. The method of claim 33, wherein rotating the insertion assembly to secure the implant to the guide plate includes engaging a flange of the outer housing with an inner recess of the guide plate.

35. The method of claim 28, further comprising securing the outer housing directly to the cranium.

36. The method of claim 28, further comprising securing the catheter in the guide plate.

37. The method of claim 36, wherein securing the catheter in the guide plate includes receiving the catheter in a transverse channel of the guide plate.

38. The method of claim 37, wherein receiving the implant in the guide plate includes aligning a cutout of the outer housing with the transverse channel of the guide plate.
